(19) United States
(12) Patent Application Publication
(10) Pub. No.: US 2005/0059633 A1
(43) Pub. Date: Mar. 17, 2005

(54) Novel glucans and novel glucansucrases derived from lactic acid bacteria

(76) Inventor: Gerritdina Hendrika Van Geel-Schuten, Driebergen (NL)

Correspondence Address: YOUNG & THOMPSON, 745 SOUTH 23RD STREET, 2ND FLOOR, ARLINGTON, VA, 22202, US

(21) Appl. No.: 10/484,218

(22) PCT Filed: Jul. 22, 2002
(86) PCT No.: PCT/NL02/00495

(30) Foreign Application Priority Data
Jul. 20, 2001 (EP) 01202752.0
Jul. 25, 2001 (EP) 01202841.1

(51) Int. Cl. A61K 31/715; C12P 19/04

(52) U.S. Cl. 514/54; 536/123.12; 435/101

(57) ABSTRACT

The invention pertains to glucans capable of being produced by glucosyltransferase activity of a lactic acid bacterium on a sucrose substrate, the glucan having an average molecular weight between 10 kDa and 1 GDa, consisting essentially of alpha (1,3)- and alpha (1,6)-linked anhydroglucose units (AGU) and to glucansucrases capable of producing these glucans from sucrose. The glucans have thickening and anti-corrosive properties. The glucans can be chemically modified.

The present invention is in the field of enzymatic production of biomolecules. The invention is particularly concerned with novel glucans derived from lactic acid bacteria, with novel glucosyl-transferases derived from such bacteria and with a process for production of new and useful glucans from sucrose.

### BACKGROUND OF THE INVENTION

Several bacteria are known to produce exopolysaccharides, i.e. polysaccharides secreted into the culture medium. Well-known examples of bacterial exopolysaccharides include xanthan from Xanthomonas campestris, gellan from Sphingomonas paucimobilis and pullulan from Aureobasidium pullulans. Lactic acid bacteria known to produce exopolysaccharides include Leuconostoc mesenteroides strains producing dextrans, α(1→6)-linked poly-anhydroglucose, and alternans i.e. poly-anhydroglucoses having alternating α(1→6) and α(1→3)-linkages, oral Streptococcus strains producing glucans responsible for dental plaque formation, and a particular Lactobacillus reuteri strain producing α(1,6)- and α(1,4)-linked anhydroglucose (Van Geel-Schutten, et al., Appl. Environ. Microbiol. (1999) 65, 3008-3014). The properties of exopolysaccharides depend on the type of monosaccharide units, the type of linkages, the degree and type of branching, the length of the polysaccharide chain, the molecular weight and the conformation of the polymers.

Argüello-Morales et al. (FEMS Microbiol. Lett. 182 (2000) 81-85) describe an alternansucrase from Leuconostoc mesenteroides NRRL B-1355. Monchois et al, (Gene 182 (1996) 23-32; FEMS Microbiol. Lett. 159 (1998) 307-315) for instance describe two different dextransucrases from Lc. mesenteroides NRRL B-1299. A method for selecting Leuconostoc mesenteroides strains that produce a high proportion of alternan to dextran is described in U.S. Pat. No. 5,789,209. The prior art does not disclose or suggest other lactic acid bacteria than Leuconostoc or Streptococcus that are capable of producing glucans having both α(1→6) and α(1→3)-linkages.

### SUMMARY OF THE INVENTION

Several lactic acid bacteria strains were found, according to the invention, to be capable of producing a particular class of glucans. These glucans have in common that their anhydroglucose units (AGU) are linked α(1,3)- and/or α(1,6)-glucosidic bonds, i.e. they are α-glucans largely or completely devoid of α(1,4)-bonds. These glucans may be of the alternan (alternating α(1,3) and α(1,6) linkages), mutan (mixed α(1,3) and α(1,6) linkages, usually α(1,3) predominant) or dextran (mainly α(1,6) linkages, some α(1,3)) type, or other type. The glucans can be produced from sucrose, using sucrase enzymes which are active in the lactic acid bacteria. They can be produced on a large scale and isolated in a commercially feasible way, as the glucans are produced outside the bacterial cell, or even in the absence of the bacteria, using isolated sucrase enzymes. The glucans are produced by food-grade strains and have interesting properties, such as prebiotic utility or thickening of water-based compositions.

The invention is concerned with these novel glucans, with the lactic acid bacterial, especially Lactobacillus strains and their enzymic proteins that produce these glucans from sucrose, as well as with methods for producing the glucans using the strains and/or their enzymes, with nucleotide sequences encoding these enzymic proteins which convert sucrose, with the use of the glucans as thickeners, prebiotics, anticorrosives, etc., and as starting materials for modified glucans.

### DESCRIPTION OF THE INVENTION

The invention pertains to Lactobacillus strains containing a glucosyltransferase (glucansucrase) capable of producing a glucan having at least 10 anhydroglucose units (AGU) having a backbone consisting essentially of α(1,3)- and/or α(1,6)-linked AGU, in the presence of sucrose. Such strains can be found among current sources of Lactobacilli, such as food sources, silage, mammalian samples etc. These strains containing the glucosyltransferases and producing the glucans can be identified by isolating Lactobacillus strains from these sources, growing them on sucrose and analysing the polysaccharide product using suitable analytical methods such as chromatography. The genes encoding these glucosyltranferases can be identified by amplifying nucleotide sequence fragments of the strain using primers based on known glucosyltransferase genes and retaining the positive strains (see examples). Several glucan-producing strains were isolated and identified from different sources and different Lactobacillus species, such as Lb. reuteri, Lb. fermentum, Lb. sake and Lb. parabuechneri or related species. The glucosyltransferases from these glucan-producing strains were also identified and, completely or partly, sequenced (see Examples).

The novel glucans of the invention are capable of being produced by glucosyltransferase (glucansucrase) activity of a lactic acid bacterium on a sucrose donor substrate. The glucans have an average molecular weight between 10 kDa and 1 GDa, and consist essentially of α(1,3)- and/or α(1,6)-linked anhydroglucose units (AGU), to which side-chains also consisting of α(1,3)- and/or α(1,6)-linked AGU may be attached.

In particular, the glucans according to the invention either comprise 15-80% of α(1,3)-linked AGU, 2-80%, especially 4-80% and more especially 15-80% of α(1,6)-linked and 2-25% of α-(1,3,6)-linked (branching) AGU, or 80-99% of α(1,6)-linked AGU and 1-20% of (1,3)-linked or α-(1,3,6)-linked (branching) AGU, in particular 1-15% of α(1,3)-linked AGU and 5-15% of α(1,3)- and α(1,3,6)-linked units taken together. Thus, the invention covers a glucan having an average molecular weight of 50 kDa to 1 MDa and comprising 25-50%, especially 29-39% of α(1,3)-linked AGU, 20-45%, especially 30-40% of α(1,6)-linked AGU, 5-25%, especially 3-13% of α(1,3,6)-linked AGU and 6-30% of terminal AGU. Furthermore, the invention pertains to a glucan having an average molecular weight of 10-50 MDa and comprising 15-26% α(1,3)-linked AGU, 30-50% of α(1,6)-linked AGU, 5-20% of α(1,3,6)-linked AGU and 5-35% of terminal AGU. Also, in another embodiment the invention covers a glucan having an average molecular weight of 1-50 MDa and comprising 40-60% of α(1,3)-linked AGU, 2-20%, especially 2-12% of α(1,6)-linked AGU, 10-25% of α(1,3,6)-linked AGU and 10-30% of terminal AGU. In yet another embodiment, the invention comprises a glucan having an average molecular weight of 10-50 MDa and comprising 80-99%, especially 88-99% and more especially 90-99% of α(1,6)-linked AGU, or 80-90% of α(1,6)- and 1-10% of α(1,3)-linked AGU, the remainder being 1,3,6 linked and terminal AGU.

The invention also concerns the enzymes originating from lactic acid bacteria, or from recombinant sources, capable of producing the glucans described above starting from sucrose. The enzymes are new and they can be classified as glucansucrases or glucosyltransferases. Their partial sequence information is given below in SEQ ID No's 1-10. More complete sequence information is given in SEQ ID No's 11-22. Proteins according to the invention comprise an amino acid sequence exhibiting at least 70%, preferably at least 80%, most preferably at least 90%, amino acid identity with any one of the amino acid sequences of SEQ ID No. 2, 4, 8, 10, 12, 14, 16, 18, 20 and 22 or of stretches of at least 221-224 amino acids thereof, or at least 100 contiguous amino acids exhibiting at least 80%, preferably at least 90%, amino acid identity with these sequences. Further preferred sequences are indicated in the description of the alignment figure given below.

The enzymes can be used as such for producing the glucans described above, or for producing oligosaccharides and polysaccharides having a similar α(1,3) and/or α(1,6) linked glucan structure. Their genes can also be incorporated in suitable host organisms, to produce alternative glucan-production systems. The invention also pertains to such recombinant, preferably food-grade microorganisms, e.g. bacteria, especially lactic acid bacteria, yeast, fungi etc., containing the genes of the glucansucrases described above and being capable of expressing the glucansucrases.

The invention also pertains to a process of producing a glucan as described above. This glucan can be produced by a Lactobacillus strain as described above, or by a recombinant micro-organism expressing the glucosyltransferase according to the invention or by an isolated glucosyltransferase according to the invention and a suitable glucose source such as for instance sucrose. The glucosyltransferase may be isolated by conventional means from the culture of a glucosyltransferase-positive lactic acid bacterium, especially a Lactobacillus species, or from a recombinant organism expressing the glucosyltransferase gene.

The glucan and the gluco-oligosaccharides produced by the Lactobacillus strains can be recovered from the culture supernatant of Lactobacillus strains described above, containing the glucosyltransferase according to the invention. The glucan can comprise at least 20, up to about 100,000 α-anhydroglucose units with the unique structure described above.

The glucan-producing enzymes according to invention, or at least the most preferred ones, are constitutive in the Lactobacillus strains, in that they are always present. This is contrast to most glucan (dextran-) producing Leuconostoc strains of the prior art, wherein the enzymes are only expressed upon growth in the presence of sucrose. This allows a more efficient production of glucans by the microorganisms of the invention.

The glucans according to invention have a variety of useful properties. They are suitable as prebiotics, and thus they can be incorporated in nutritional or pharmaceutical compositions intended for improving the condition of the gastrointestinal tract. For this purpose, they can be used as such or in the form of their oligosaccharides. They can also be combined with other poly- or oligosaccharides, such as fructans, galactans, xylans, arabinans, mannans, indigestible glucans and hetero-oligosaccharides, or with probiotic micro-organisms, including the lactic acid bacteria from which the glucans originate, resulting in synbiotic compositions. The glucans and their shortened homologues are also useful as bioactive agents, e.g. as immunomodulators, anti-ulcer agents and cholesterol-lowering agents.

The glucans are also useful as thickening agents. As such they can be incorporated in foodstuffs such as beverages, sauces, dressings, dairy products, in amounts of from 1 g/l to about 100 g/l, especially about 10 to 50 g/l.

The glucans of the invention are furthermore useful as anticorrosion agents, for example for the protection of ship hulls. For that purpose, they may be applied in the form of solutions or suspensions, by spraying, coating, dipping and other techniques known in the art of corrosion control.

The glucans can be used as such. They can also be modified by physical or chemical means. Suitable examples of chemical modification include oxidation, especially 2,3- or 3,4-oxidation using periodate or hypohalite, in glucans having α-1,6 linkages, or 6-oxidation using nitroxyls with peracid or hypohalite in glucans having α-1,3 linkages. Hypohalite oxidation resulting in ring-opened 2,3- or 3,4-dicarboxy-anhydroglucose units (see e.g. EP-A-427349), while periodate oxidation results in ring-opened 2,3- or 3,4-dialdehyde-anhydroglucose units (see e.g. WO 95/12619), which can be further oxidised to (partially) carboxylated units (see e.g. WO 00/26257). Nitroxyl-mediated oxidation using hypochlorite or a peracid results in 6-aldehyde- and 6-carboxy-anhydroglucose units (see e.g. WO 95/07303).

The oxidised glucans have improved water-solubility, altered viscosity and a retarded fermentability and can be used as metal-complexing agents, detergent additives, strengthening additives, bioactive carbohydrates, emulsifiers and water binding agents. They can also be used as starting materials for further derivatisation such as cross-linking and the introduction of hydrophobes. Oxidised glucans coupled to proteins can be used as emulsifiers and stabilisers. The oxidised glucans of the invention preferably contain 0.05-1.0 carboxyl groups, more preferably 0.2-0.8 carboxyl groups per anhydroglucose unit, e.g. as 6-carboxyl groups on 1,3-linked units.

When modified glucans with high proportion of carboxyl groups are desired, two oxidation processes can be combined or an oxidation can be combined with e.g. carboxymethylation (see below). Thus, an α-(1,3/1,6)-glucan having a degree of substitution (DS) for carboxyl groups between 0.3 and 1.0 can be conveniently prepared by first nitroxyl-mediated oxidation, resulting in 1,3-substituted units being oxidation to glucuronic acid units, followed by e.g. periodate and chlorite oxidation, resulting in 1,6-substituted units* being converted to ring-opened dicarboxy-substituted units. The order of processes can also be inverted, or one oxidation process, such as nitroxyl-mediated 6-oxidation can be combined with carboxymethylation. Also, by appropriate adaptation of the oxidation processes mixed aldehyde-containing and carboxyl-containing polymers can be obtained.

Other useful modifications are alkylation, acylation, hydroxyalkylation, aminoalkylation, carboxyalkylation, phosphorylation, sulphatation, as well as physical and chemical crosslinking. Phosphorylation (see: O. B. Wurzburg (1986), Modified Starches: properties and uses. CRC Press Inc., Boca Raton, 97-112) can be achieved by dry heating glucans with a mixture of monosodium and disodium hydrogen phosphate or with tripolyphosphate. The phosphorylated glucans are suitable as wet-end additives in papermaking, as binders in paper coating compositions, as warp sizing-agents, and as core binders for sand molds for metal casting. Acylation, especially acetylation or propionylation using acetic or propionic anhydride respectively, results in products suitable as bleaching assistants and for the use in foils. Acylation with e.g. alkenyl succinic anhydrides or (activated) fatty acids results in surface-active products suitable as e.g. surfactants, emulsifiers, and stabilisers. Crosslinking e.g. by coupling oxidised derivatives, or by reaction with a crosslinking agent such as triphosphoric acid, epichlorohydrine or a dialdehyde, can be used to adjust the physical properties of the glucans, e.g. to enhance their water-binding or thickening capacities.

Hydroxyalkylation is commonly performed by base-catalysed reaction with alkylene oxides, such as ethylene oxide, propylene oxide or epichlorohydrin; the hydroxyalkylated products have improved solubility and viscosity characteristics. Carboxymethylation is achieved by reaction of the glucans with monochloroacetic acid or its alkali metal salts and results in anionic polymers suitable for various purposes including cystallisation inhibitors, and metal complexants. Amino-alkylation can be achieved by reaction of the glucans with alkylene-imines, halo-alkyl amines or amino-alkylene oxides, or by reaction of epichlorohydrine adducts of the glucans with suitable amines. These products can be used as cationic polymers in a variety of applications, especially as a wet-end additive in paper making to increase strength, for filler and fines retention, and to improve the drainage rate of paper pulp. Other potential applications include textile sizing and wastewater purification. The above mentioned modifications can be used either separately or in combination depending on the desired product. Furthermore, the degree of chemical modification is variable and depends on the intended use. If necessary 100% modification, i.e. modification of all anhydroglucose units can be performed. However, partial modification, e.g. from less than 1 (e.g. 0.2) modified anhydroglucose unit per 100 units up to higher levels, will often be sufficient in order to obtain the desired effect.

Another suitable type of derivatives is formed by hydrolysates of the present glucans. Hydrolysis can be performed in a controlled manner in a way known per se, using e.g. dilute acid or glucanolytic enzymes, especially α-1,3-glucanases or α-1,6 glucanases. Hydrolysis results in polysaccharides of reduced chain length (degree of polymerisation, DP, of more than 20) or oligosaccharides (DP of less than 20).

The invention also relates to gluco-oligosaccharides containing the characteristic structure of the glucan described above. These can be produced using an isolated glucansucrase according to the invention or a Lactobacillus strain, or a recombinant micro-organism containing (a part of) a glucosyltransferase according to the invention. Gluco-oligosaccharides thus produced can be used as prebiotics and probiotics. The production of the gluco-oligosaccharides is different from the glucan synthesis reaction. In addition to sucrose, the substrate of the glucansucrase, an acceptor molecule such as maltose or lactose can be used as an acceptor, to synthesise oligosaccharides. Consecutive attachment of glucose units in a manner determined by the particular glucansucrase results in α(1,3)- and/or α(1,6)-linked gluco-oligosaccharides, the chain length of which can be determined by selecting the appropriate reaction conditions. Longer reaction times, higher sucrose levels and lower acceptor levels will usually result in relatively long chains, e.g. having a degree of polymerisation (DP) of more than 10, up to several hundreds if desired, while shorter reaction times, lower sucrose levels and higher acceptor levels will result in relatively short chains, e.g. with a DP from about 3 up to 10 or higher. Another way of producing gluco-oligosaccharides is by hydrolysis of the glucan described above. This hydrolysis can be performed by known hydrolysis methods such as enzymatic hydrolysis with enzymes such as amylase, dextranase or pullulanase or by acid hydrolysis. The produced gluco-oligosaccharides contain at least one 1,6- or one 1,3-glucosidic link to be used as prebiotics.

The invention also relates to a probiotic or synbiotic composition containing a Lactobacillus strain capable of producing a glucan and/or gluco-oligosaccharide according to the invention. The strain may also produce another poorly digestible poly- or oligosaccharide, such as a fructan. The probiotic or synbiotic compositions of the invention may be directly ingested with or without a suitable vehicle or used as an additive in conjunction with foods. They can be incorporated into a variety of foods and beverages including, but not limited to, yoghurts, ice creams, cheeses, baked products such as bread, biscuits and cakes, dairy and dairy substitute foods, confectionery products, edible oil compositions, spreads, breakfast cereals, juices and the like.

Furthermore, the invention pertains to a process of improving the microbial status in the mammalian colon comprising administering an effective amount of a Lactobacillus strain capable of producing a glucan and/or gluco-oligosaccharide according to the invention. Furthermore, a process of improving the microbial status of the mammalian colon comprising administering an effective amount of a glucan or gluco-oligosaccharide according to the invention is also a part of the present invention.

### EXAMPLES

General

The various lactic acid bacterial strains were isolated from a variety of sources, including fermented foods, the gastrointestinal tract of various human or animal species, and silage.

### Example 1

### Identification and Nucleotide Sequence of Glucansucrase/Glucosyltransferase Genes from Lactobacilli

The glucansucrase genes were identified by amplification with PCR using degenerated primers (GTFrev, 5′ ADRTC NCCRT ARTAN AVNYK NG 3′ and GTFforw, 5′-GAYAAYWSNA AYCCNRYNGT NC-3′; N=A, C, G or T, Y=T or C, K=G or T, W=A or T, S=C or G, R=A or G), based on conserved amino acid sequences of different published glucansucrase genes. An amplification product with the predicted size of about 660 bp was obtained and cloned in Escherichia coli Top 10 using pCR-XL-TOPO (Invitrogen). Sequence analysis confirmed that part of a gtf gene had been isolated. The 660 bp amplified was used to design primers for inversed PCR. For inverse PCR chromosomal DNA was digested with 10 different enzymes ligated, yielding circular DNA molecules. PCR with the diverging primers with the circular ligation products as template yielded amplicons of various sizes, those products were cloned into pCR-XL-TOPO (Invitrogen) and sequenced (GATC, Konstanz, Germany). If necessary additional inverse PCR reactions were carried out to obtain the complete gene(s). Both strands of the entire glucansucrase genes were sequenced twice.

### Example 2

### Isolation and Identification of α-(1,6) Glucan and a Glucansucrase from Lactobacillus reuteri Strain 180

L. reuteri strain 180 was deposited as LMG P-18389 at the BCCM/LMG Culture Collection at Gent, Belgium. The strain was grown in 18 litres of MRS-s medium (in g per kg): yeast extract (22), sodium acetate trihydrate (5), sodium citrate dihydrate (2.42), ammonium chloride (1.32), dipotassium hydrogen phosphate (2), magnesium sulphate heptahydrate (0.2), manganese sulphate heptahydrate (0.05), sorbitan mono-oleate (1), vitamins (in mg per kg: B1: 14.4, B2: 3.6, B3: 72, H 0.216), sucrose (100), tap water (remainder), for 21 h at 37° C. under anaerobic conditions (pH 5.5). See also: Van Geel-Schutten et al., Appl. Microbiol. Biotechnol. (1998) 50, 697-703. During growth, 13 g/l polysaccharide was produced. This polysaccharide was isolated as described in the reference cited above. The monosaccharide composition of the polysaccharide was determined by hydrolysis of the soluble part of the polysaccharide and high-performance anion-exchange chromatography. It was characterised as a glucan. This glucan was not formed when the strain was grown on glucose instead of sucrose. Methylation analysis (Van Geel-Schutten et al. 1999) revealed the presence of 17-24% α(1,3)-linked glucosyl units, 34-44% of α(1,6)-linked glucosyl units, 7-15% of α(1,3,6)-linked glucosyl units and 7-35% of terminal glucosyl units. The average molecular weight of the glucan was determined to be 3.6×10⁷ Da and the Rg was 45 nm.

The average molecular weight of the polysaccharide was established using the SEC-MALLS system: 0.0522 g of the glucan was dissolved in 10 ml DMSO/water (90/10) and heated for 1 hour at 80° C., filtered through a 0.45 μm filter and injected on the SEC-MALLS system and analysed using the following conditions.

| | |
|---|---|
| Eluent: | DMSO/water (90/10) with 0.1 M NaNO₃ |
| Flow rate: | 0.5 ml/min |
| Injection volume: | 0.247 ml |
| Column: | PLgel Guard, mixed-A and mixed-D |
| Temperature: | 90° C. |

Detection: MALLS (DAWN-DSP), 50° C., A₂=0, dn/dc=0.074, F2 cell, RI; SDS PAGE followed by PAS-staining (Van Geel-Schutten et al. 1999) revealed the presence of an extracellular sucrase with a molecular weight of about 190 kDa. Part of the gene encoding the sucrase enzyme was isolated using PCR techniques and sequenced. On the deduced amino acid sequence of the fragment, high homologies were found with other glucansucrases. This partial sequence information is given in SEQ ID No. 1 (DNA) and 2 (protein). Full sequence information is given in SEQ ID No's. 11 and 12.

The glucan produced by L. reuteri strain 180 has been tested for application on ship hulls for the prevention of corrosion (see Example 8).

### Example 3

### Isolation and Identification of α-(1,6/1,3) Glucan and a Glucansucrase from Lactobacillus reuteri Strain ML1

L. reuteri strain ML1, deposited as LMG P-20347 at the BCCM/LMG Culture Collection at Gent, Belgium, was grown overnight under anaerobic conditions at 37° C. on MRS supplemented with sucrose (see Example 2). The cells were removed by centrifugation and two volumes of ethanol were added to the supernatant. The precipitated polysaccharides were harvested by centrifugation and resuspended in 2-3 liters of demi water and precipitated again with two volumes of ethanol. The glucan produced by this strain (7 g) was characterised by methylation analysis and monosaccharide composition analysis as described in Example 2. The polymer was found to consist of 48-53% of α(1-3) linked glucosyl units, 3-8% of α(1-6) linked glucosyl unit; 12-20% of α(1-3-6) linked glucosyl units (branching units) and 20-30% of 1-linked (terminal) glucose units. The glucans were not produced during growth on glucose. The average molecular weight of the polysaccharide was established to be 7.6×10⁶ Da using the SEC-MALLS system as described in example 2. These were the first examples of the production of mutan-like polymers by lactobacilli. The glucan produced by L. reuteri strain ML1 has been tested for application as anticorrosive agent and showed excellent utility for the prevention of corrosion e.g. on ship hulls.

SDS PAGE followed by PAS-staining (Van Geel-Schutten et al. 1999) revealed the presence of an extracellular sucrase with a molecular weight of about 190 kDa. It was found that this strain produces two glucansucrases. Sequence information for these sucrase is given in SEQ ID No's 13 and 14 (ML1) and 15 and 16 (ML4).

### Example 4

### Isolation and Identification of α-(1,6/1,3) Glucan and a Glucansucrase from Lactobacillus Strain LB 33

A new Lactobacillus strain was obtained and was deposited as LMG P-20349. The strain was identified by 16S rRNA to be most closely related to Lactobacillus parabuchneri. The strain grown overnight on MRS supplemented with sucrose under anaerobic conditions at 37° C. (see Example 2). 420 gram of glucan was produced. The glucan produced by this strain is not produced during growth on glucose.

Methylation analysis (see Example 2) revealed that the polymer consists of equal amounts of 29-39% of α(1-3) linked glucosyl units, 30-40% of α(1-6) linked glucosyl units, 3-13% of α(1-3-6) linked glucosyl units (branching units) and 15-30% of 1-linked (terminal) glucose units.

The average molecular weight of the polysaccharide was established to be 2×10⁵ Da, using the SEC-MALLS system as described in Example 2.

By PCR with degenerated primers part of a sucrase type of glucosyl-transferase could be isolated indicating that the glucan is produced by a sucrase. This confirms the result that the glucan is produced during growth on sucrose and not on glucose. Part of the sucrase encoding gene was sequenced. On the deduced amino acid level high homologies were found with alternan sucrase from Leuconostoc mesenteroides. This indicates that the enzyme responsible for the glucan synthesis in L. brevis is the first alternan sucrase found in other bacteria than Leuconostoc. This partial sequence information is given in SEQ ID No. 3 (DNA) and 4 (protein). Full sequence information is given in SEQ ID No's. 17 and 18, respectively.

The glucan produced by this strain has thickening properties.

### Example 5

### Isolation and Identification of α-(1,6) Glucan and a Glucansucrase from Leuconostoc Strain 86

A new strain was obtained from silage and was deposited as LMG P-20350. The strain was identified by 16S rRNA to be a new Leuconostoc strain, most closely related to Leuconostoc citreum. The strain grown overnight on MRS supplemented with sucrose under anaerobic conditions at 37° C. (see Example 2). 416 gram of glucan was produced. Methylation analysis of the glucan obtained revealed that more than 90% of the glucose units was linked through an α(1,6) bond, identifying the polysaccharide as a dextran. The molecular weight of the glucan (determined as described in Example 2) was 3-4×10⁷ Da and the Rg was 40 nm. The glucan is not produced during growth on glucose.

By PCR with degenerated primers 3 different fragments with part of a sucrase type of glucosyl-transferase could be isolated indicating that the glucan is produced by a sucrose and that possibly 3 sucrases are present in this strain. This confirms the result that the glucan is produced during growth on sucrose and not on glucose. Part of the sucrase encoding gene was sequenced. On the deduced amino acid level high homologies were found with DSRC and DSRB (fragment 1), alternan sucrase (fragment 2) and DSRA (fragment 3) from Leuconostoc mesenteroides. The sequence information is given in SEQ ID No's 5-10, Leuconostoc citreum, to which this new strain is most closely related, is not reported to produce dextran. The glucan produced by strain 86 has thickening properties.

### Example 6

### Identification of α-(1,6/1,3) Glucan and a Glucansucrase from Lactobacillus sake KG 15

Strain KG 15 was obtained from silage and was deposited as LMG P-21583. It was identified by 16S rRNA as L. sake. The strain was grown and the polysaccharide was recovered as described in example 2. The molecular weight of the polysaccharide was determined to be 4.7 10⁷ Da (SEC MALLS) and the Rg was 92 nm. Methylation analysis (GC) revealed that the glucan produced by this strain is a largely linear dextran containing 4% terminal glucose units, 86% of α(1,6) linked glucosyl units, 2% of α(1,3) linked glucosyl units and 8% α(1,3,6) disubstituted glucose units (branching points). The glucansucrase of this strain was sequenced (see SEQ ID No. 19 and 20).

### Example 7

### Identification of α-(1,6/1,3) Glucan and a Glucansucrase from Lactobacillus fermentum KG 3

Strain KG 3 was obtained from silage and was deposited as LMG P-21584. It was identified by 16S rRNA as L. fermentum. The strain was grown and the polysaccharide was recovered as described in example 2. The molecular weight of the polysaccharide was determined to be 2.4 10⁷ Da (SEC MALLS) and the Rg was 107-119 nm. Methylation analysis (GC) revealed that the glucan produced by this strain is a largely linear dextran containing 3% terminal glucose units, 84% of α(1,6) linked glucosyl units, 8% of α(1,3) linked glucosyl units and 5% α(1,3,6) disubstituted glucose units (branching points). The glucansucrase of this strain was sequenced (SEQ ID No's 21 and 22).

### Example 8

### Anticorrosion Properties of Glucans

Plain carbon steel sheets of 1 cm² embedded in an epoxy matrix were exposed to a slightly corrosive medium (150 ml of 0.1 M LiClO₄) with or without the addition of a bacterial polysaccharide (0.2 g) for several days. The sheets were then examined visually and electrochemically from time to time. The corrosion potential (E_{corr} in mV with reference to Ag/AgCl) and polarisation resistance (Rₚ in kΩ/cm²) are both a measure of the anti-corrosion effect. After an initial adaptation of 3-10 hours, these parameters attained a stable value. The experiments were carried with a heteropolysaccharide from Lactobacillus sake, and a homopolysaccharide of the invention (from LB 180 according to example 4), as well as without polysaccharide. The results are summarised in the table below. It follows that the anti-corrosion properties of the glucan of the invention are superior. It was found that the homopolysaccharide of ML 1 (example 3) has at least equal anticorrosion performance as the LB 180 polysaccharide.

TABLE
Corrosion experiments

| | | | E_{corr} | |
|---|---|---|---|---|
| | type of poly- | aspect of treated | (mV vs. | Rₚ |
| organism | saccharide | sheet | Ag/AgCl) | (kΩ/cm²) |
| control | — | corrosion | −700 | 1.5 |
| Lb. sake | hetero- | localised corrosion | −600 | 4.5 |
| | polysaccharide | | | |
| Lb. 180 | α-glucan | thin black layer | −200 | 7.0 |

### Example 9

### Modification of α-1,3/1,6-Glucan by Oxidation

One gram (6.15 mmol of anhydroglucose units) of the α-1,3/1,6-glucan produced by strain LB 33 (example 4) is resuspended in 100 ml water. Next, 2,2,6,6-tetramethyl-piperidine-1-oxyl (TEMPO; 0.01 g, 0.065 mmol) and sodium bromide (100 mg, 1 mmol) are added and the suspension is cooled to 0° C. The reaction can also be performed without bromide. A solution of hypochlorite (3 ml, 15% solution, 6.3 mmol) of pH 10.0 (0° C.) is added. The pH is kept constant by addition of 0.1M NaOH. After 1 hr, the solution is poured into 150 ml 96% ethanol, causing the product to precipitate. The white precipitate is centrifuged, resuspended in ethanol/water (70/30 v/v) and centrifuged again. Next, the precipitate is resuspended in 96% ethanol, centrifuged and dried. The uronic acid content is determined by means of the uronic acid assay according to Blumenkrantz and Abdoe-Hansen (Anal. Biochem. 54 (1973), 484). A calibration curve was generated using polygalacturonic acid (5, 10, 15 and 20 μg). With this calibration curve the uronic acid content in a sample of 20 μg of the product is determined. The major part of 6-hydroxyl groups have been oxidised to carboxyl groups.

### Example 10

### Construction of Plasmids for Expression of the Glucansucrase Genes in E. coli

Two primers were designed with appropriate restriction sites; the C-terminal primer contained in all cases a His-tag. The PCR products were first cloned in pCR-XL-TOPO. The PCR products were removed from pCR-XL-TOPO using the appropriate enzymes and ligated in the appropriate sites of an expression vector (e.g pET15b (Novagen)).

For the expression of part of the glucosyltransferase gene of LB 180 (for better expression, the N-terminal region encoding the N-terminal variable domain of the glucansucrase, was not cloned) in E. coli, a PCR reaction was performed using Forw180 (5′-GATGCATGAG CTCCCATGGG CATTAACGGC CAACAATATT ATTATTGACC C-3′) containing SacI (bold) and NcoI (underlined) sites, and Rev180 (5′-ATATCGATGG GCCCCGGATC CTATTAGTGA TGGTGATGGT GATGTTTTTG GCCGTTTAAA TCACCAGGTT TTAATGG-3′), containing ApaI (bold), BamHI (underlined) and a 6×His-tag (italics) as primers. The PCR product was cloned in pCR-XL-TOPO. The PCR product was removed from pCR-XL-TOPO using NcoI/BamHI and ligated in the coresponding sites of pET15b (Novagen). The resulting plasmid (pET15b180) containing part of the glucansucrase gene of 704 amino acids encoding a glucansucrase without the variable N-terminal domain was transformed to E. coli B121 DE3 star (Invitrogen).

Cells of E. coli harbouring the pET15b180 were harvested by centrifugation after 16 h of growth under aerobic conditions at 37° C. The pellet was washed with 50 mM sodium acetate buffer pH 5.5 containing 1 mM CaCl₂ and 1% (v/v) Tween 80 and the suspension was centrifuged again. Pelleted cells were resuspended in with 50 mM sodium acetate buffer pH 5.5 containing 1 mM CaCl₂ and 1% (v/v) Tween 80, and 7.2 mM β-mercapto-ethanol. Cells were broken by sonication and cell debris and intact cells were removed by centrifugation for 15 minutes at 4° C. at 14,000 rpm (Eppendorf). The resulting cell free extract was used as enzyme source to produce high molecular weight glucans from sucrose in 50 mM sodium acetate buffer pH 5.5 containing 1 mM CaCl₂ and 1% (v/v) Tween 80 and 10 g/l sucrose. After 16 hours of incubation, the glucans were isolated using ethanol precipitation. When cell free extracts of E. coli B121 DE3 star (Invitrogen) harbouring the plasmid pET15b (without insert) were used as enzyme source, no glucans were produced from sucrose.

Sequence Information

SEQ ID No's 1 and 2 give the nucleotide and amino acid sequence, respectively, of a part of the glucansucrase from strain Lb180 as originally determined (Example 2). The partial sequence shows 53% (199/223) sequence identity and 68% similarity with dextransucrase DSRB742 of Leuconostoc mesenteroides (Lc. mes.), with 2 gaps (between amino acids F172 and N173), and 52% identity with some other dextransucrases and alternansucrases of Lc. mes.

SEQ ID No's 3 and 4 give the nucleotide and amino acid sequence, respectively, of a part of the glucansucrase from strain Lb 33 as originally determined (Example 4). The partial sequence shows 63% (143/224) sequence identity and 75% similarity with dextransucrase DSRB742 of Lc. mes. with 1 gap.

SEQ ID No's 5 and 6 give the nucleotide and amino acid sequence, respectively, of a part of a glucansucrase (86-1) from strain Lc 86 (Example 5). The partial sequence shows 98% (219/223) sequence identity and 99% similarity with dextransucrase DSRB742 of Lc. mes.

SEQ ID No's 7 and 8 give the nucleotide and amino acid sequence, respectively, of a part of another glucansucrase (86-5) from strain Lc 86 (Example 5). The partial sequence shows 55% (123/223) sequence identity and 68% similarity with dextransucrase DSRB742 of Lc. mes., with 2 gaps (between amino acids M128 and R129 and between D162 and H163), and 51-56% identity with some other dextransucrases and alternansucrases of Lc. mes.

SEQ ID No's 9 and 10 give the nucleotide and amino acid sequence, respectively, of another glucansucrase (86-8) from strain Lc 86 (Example 5). The partial sequence shows 61-68% sequence identity and 74-78% similarity with dextransucrases and alternansucrases (including dextransucrase DSRB742 ) of Lc. mes.

SEQ ID No's 11 and 12 give the nucleotide and amino acid sequence, respectively, of the glucansucrase of strain Lb180 (Example 2). The sequence shows 1322/1768 (74%) sequence identity and 1476/1768 (82%) similarity with 15/1768 gaps with glucansucrase from Lb. reuteri LB 121 as disclosed in WO 01/90372. The −35 and −10 sites TTGAAA and TATAA are located at nucleotide positions 561 and 599, respectively. The ribosome binding site (RBS) GAAGGAG is at 574 and the start codon ATG at 587. Inverted repeats AAGCAGCTC and GAGCTGCTT are at 6025 and 6051. Possible stop codons (TAA, TAG, TGA) are indicated with an * (5963).

SEQ ID No's 13 and 14 give the nucleotide and amino acid sequence, respectively, of the glucansucrase I from strain ML1 (Example 3). The sequence shows 1327/1775 (74%) sequence identity and 1465/1775 (81%) similarity with 17/1775 gaps with glucansucrase from Lb. reuteri LB 121 as disclosed in WO 01/90372, and 43-44% sequence identity and 57-58% similarity with dextransucrases of Lc. mes. and 47% sequence identity and 61% similarity with an alternansucrases of Lc. mes. The RBS AAGGAGA is at 31 and the start codon ATG is at 43. A stop codon TAG is at 5356.

SEQ ID No's 15 and 16 give the partial nucleotide and amino acid sequence, respectively, of a second glucansucrase from strain ML1 (ML4) (Example 3). The sequence shows 301/817 (36%) sequence identity and 427/817 (51%) similarity with 12/817 gaps with glucansucrase from Lb. reuteri LB 121 as disclosed in WO 01/90372, and 38% sequence identity and 53% similarity with glucosyltransferase of Streptococcus mutans.

SEQ ID No's 17 and 18 give the partial nucleotide and amino acid sequence, respectively, of the glucansucrase from strain LB 33 (Example 4). The sequence shows 59% sequence identity and 71% similarity with several known dextransucrases of Lc. mes. and 53% sequence identity and 67% similarity with other known dextransucrases (including dextransucrase DSRB742) of Lc. mes.

SEQ ID No's 19 and 20 give the nucleotide and amino acid sequence, respectively, of the glucansucrase from Lb. strain KG 15 (Example 6). The sequence shows 496/1111 (44%) sequence identity and 637/1111 (56%) similarity with 71/1111 gaps with glucansucrase from Lb. reuteri LB 121 as disclosed in WO 01/90372, and 57-59% sequence identity and 70% similarity with several dextransucrases (including dextransucrase DSRB742) of Lc. mes. The −35 and −10 sites TTGGAC and TATTAT are located at nucleotide positions 477 and 502, respectively. The RBS GAAAGGA is at 593 and the start codon ATG at 608. A stop codon TAG is 5393. Inverted repeats AAAACAACCCCC and GGGGTTGTTTTT are at 5497 and 55 31 (−10.7 kcal/mole).

SEQ ID No's 21 and 22 give the partial nucleotide and amino acid sequence, respectively, of the glucansucrase from Lb. strain KG 3 (Example 7). The sequence shows 58 sequence identity and 71% similarity with known dextransucrases (including dextransucrase DSRB742) of Lc. mes.

### DESCRIPTION OF THE FIGURE

FIG. 1 depicts an amino acid sequence alignment of glucosyltransferases (GTF) according to the invention. It shows the partial sequences of the GTF of Lb 180 (first line, starting with amino acid 216 of SEQ ID No. 12); GTF of ML1 (second line, starting with amino acid 15 of SEQ ID No. 14), GTF of Lb 33 (third line, starting with amino acid 222 or 243 of SEQ ID No. 18); GTF of KG15 (fourth line, starting with amino acid 567 of SEQ ID No. 20) and GTF of KG3 (fifth line, starting with amino acid 1 (LMAAF) of SEQ ID No. 22); and a GTF according to the invention of a Lb. reuteri strain “104” (sixth line, 1 (WPNTV)-525). The alignment is not necessarily the best fit according to automated alignment programs, but is intended to define the enzymes of the invention.

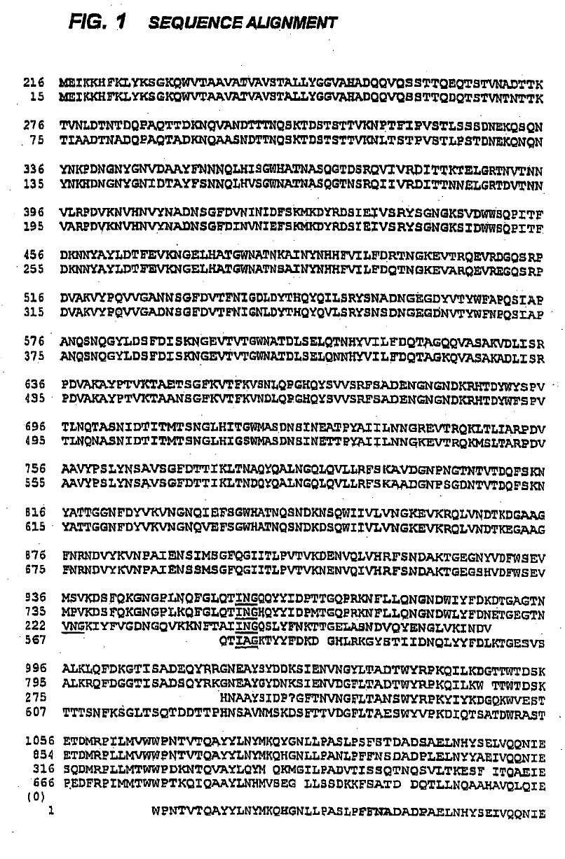

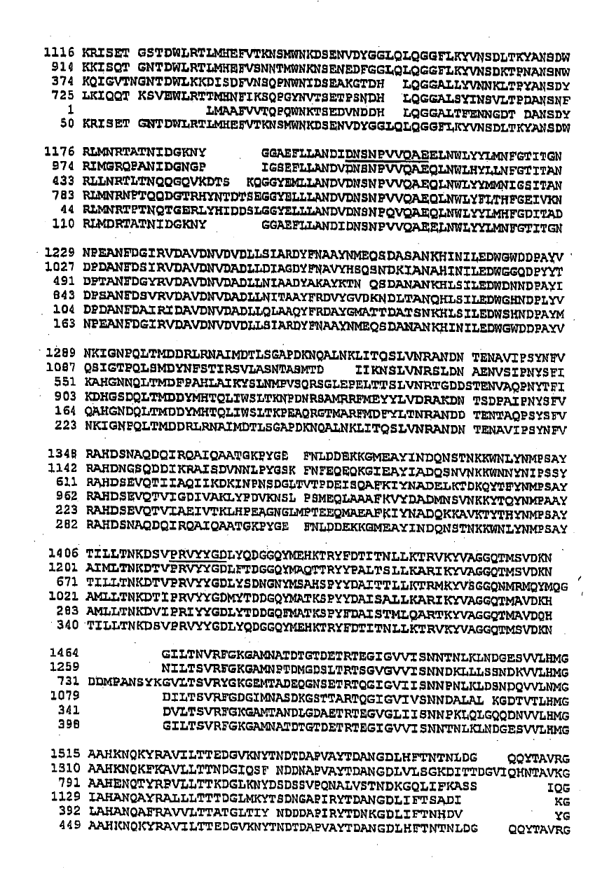

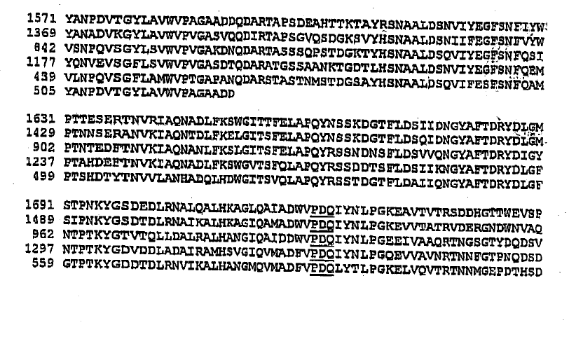

The invention not only covers amino acid sequences shown in this FIGURE, but also sequences wherein amino acids of a given sequence in the FIGURE are exchanged with the corresponding amino acids (including gaps) of another sequence of the FIGURE. This applies to stretches of at least 100 amino acids having at least 80%, preferably at least 90% identity with any of the sequences of the FIGURE, or of the sequences listings given separately. It especially applies to the stretch of amino acids between the consensus peptides DNSN and YYGD (from 1202 to 1422 of SEQ ID No 12). Especially preferred are sequences comprising the active core of the enzymes, which are present between the consensus peptides INGQ and VPDQ (from 957 to 1724 of SEQ ID No 12), with preferably at least 70% identity with any one of the core sequences given. A preferred non-identity with a given sequence is an exchange with the corresponding amino acids of another sequence. Especially preferred sequences are those where an amino acid at a given position is shared between at least 2, in particular at least 3, of the sequences of the FIGURE. Most preferred are those sequences in which one of those consensus sequences is that of the GTF of Lb180, ML1 or Lb33 (fist three lines). The N-terminal part upstream of the core (shown in the FIGURE for GTF 180 and GTF ML1 only), or the C-terminal part downstream of the core (not shown in the FIGURE) may be wholly or partly present or may be absent.

1-20. (cancelled)

21. A process of producing a glucan having at least 10 anhydroglucose units, having a backbone consisting essentially of α(1,3)- and/or α(1,6)-linked anhydroglucose units (AGU), comprising subjecting sucrose to the activity of a glucosyltransferase produced by a Lactobacillus strain capable of producing α(1,3)- and/or α(1,6)-linked glucans, or to the Lactobacillus strain capable of expressing the glucosylfransferase.

22. A Lactobacillus strain capable of producing, in the presence of sucrose, a glucan having at least 10 anhydroglucose units (AGU) having a backbone consisting essentially of α(1,3)- and/or α(1,6)-linked AGU.

23. A glucan capable of being produced by glucosyltransferase activity of a lactic acid bacterium on a sucrose substrate, the glucan having an average molecular weight between 10 kDa and 1 GDa, and having a backbone consisting essentially of α(1,3)- and α(1,6)-linked anhydroglucose units (AGU).

24. The glucan according to claim 23, which has an average molecular weight between 10 kDa and 50 Mda.

25. The glucan according to claim 23, which is capable of being produced by glucosyltransferase activity of a Lactobacillus species.

26. The glucan according to claim 25, comprising 15-80% of α(1,3)-linked AGU, 2-80% of α(1,6)-linked AGU, and 2-25% of α-(1,3,6)-linked AGU.

27. The glucan according to claim 26, having an average molecular weight of 50 kDa-1 MDa and comprising 30-45% of α(1,3)-linked AGU, 30-45% of α(1,6)-linked AGU, and 3-13% of α(1,3,6)-linked AGU.

28. The glucan according to claim 26, having an average molecular weight of 10-50 MDa and comprising 15-26% α(1,3)-linked AGU, 30-50% of α(1,6)-linked AGU, 5-20% of α(1,3,6)-linked AGU.

29. The glucan according to claim 26, having an average molecular weight of 1-50 MDa and comprising 45-60% of α(1,3)-linked AGU, 4-10% of α(1,6)-linked AGU, and 10-20% of α(1,3,6)-linked AGU.

30. A glucan capable of being produced by glucosyltransferase activity of a lactic acid bacterium on a sucrose substrate, having an average molecular weight of 10-50 MDa and comprising 80-99% of α(1,6)-linked AGU and 0-15% of α(1,3)-linked AGU.

31. A protein having glucosyltransferase activity, capable of producing, in the presence of sucrose, a glucan according to claim 23.

32. The protein according to claim 31, comprising an amino acid sequence of at least 100 amino acids, the sequence exhibiting at least 80% amino acid identity with any one of the amino acid sequences of SEQ ID No. 2, 4, 8, 10, 12, 14, 16, 18, 20 and 22, and/or having a stretch of 100 amino acids having at least 90%, amino acid identity with any one of the said amino acid sequences.

33. The protein according to claim 31, comprising an amino acid sequence having at least 99% amino acid identity with the amino acid sequence of SEQ ID No. 6, and/or having a stretch of 100 amino acids having 100% amino acid identity with the amino acid sequence of SEQ ID No. 6.

34. A nucleic acid sequence encoding the protein according to claim 31.

35. A recombinant host cell containing one or more copies of a nucleic acid construct comprising a nucleic acid sequence according to claim 34 and capable of expressing a protein having glucosyl-transferase activity.

36. A Lactobacillus strain, capable of producing the glucan according to claim 23.

37. The Lactobacillus strain according to claim 36, corresponding to strain 33, 180 or ML1 as described herein.

38. A Leuconostoc strain, capable of producing the glucan according to claim 30.

39. The Leuconostoc strain according to claim 38 corresponding to strain 86, deposited under accession number LMG P-20350.

40. A chemically modified glucan, which is obtained by 2,3-oxidation, 6-oxidation, phosphorylation, acylation, alkylation, hydroxyalkylation, carboxymethylation, aminoalkylation of one or more AGU of a glucan according to claim 23.

41. A method of thickening a water-based composition, coprising incorporating in said composition the glucan according to claim 23.

42. A method of controlling corrosion of a substrate, comprising applying onto the substrate the glucan according to claim 23.

43. A method of improving the condition of the gastrointestinal tract, modulating immune properties, controlling ulcers or lowering cholesterol levels, comprising administering an effective amount of the glucan according to claim 23.

44. A method of improving the condition of the gastrointestinal tract, comprising administering an effective amount of a Lactobacillus bacterium capable of producing the glucan according to claim 23 as a probiotic agent, or together with an indigestible glucan, as a synbiotic agent.

45. A nutritional or pharmaceutical composition comprising the glucan of claim 23 as a prebiotic or as a bioactive agent.


SEQUENCE LISTING (30 sequences)

SEQ ID NO: 1 - DNA, 665 bp, Lactobacillus reuteri
gataatacgaatccggtggtgcaagctgaagaattaaactggctttactatttaatgaatttcggtacaattacaggaaataatcctgaagctaattttgatggtattcgagtggatgctgttgataatgtagatgttgacttattgagtattgcacgtgattactttaatgcagcatataacatggagcaaagtgatgccagtgctaataagcacattaatattttggaagattggggatgggatgatcctgcttatgtaaataagattggaaatcctcaattaacaatggatgatcgtttacgaaatgcaattatggatacattatcaggagcacctgataaaaaccaagcattgaataaattaattactcagtcattagtaaatcgtgctaatgataatactgaaaacgcggttattccaagctataattttgttcgagcacatgatagtaatgctcaagaccaaattcgtcaggctattcaagctgcaactggaaaaccatatggcgaatttaacttagatgatgaaaagaagggtatggaagcatatattaatgatcagaattctactaataagaagtggaatctttacaatatgccttctgcttatactattcttctaacaaataaagattcagttccccacgtctactatggcgacat

SEQ ID NO: 2 - protein, 221 aa, Lactobacillus reuteri
DNTNPVVQAEELNWLYYLMNFGTITGNNPEANFDGIRVDAVDNVDVDLLSIARDYFNAAYNMEQSDASANKHINILEDWGWDDPAYVNKIGNPQLTMDDRLRNAIMDTLSGAPDKNQALNKLITQSLVNRANDNTENAVIPSYNFVRAHDSNAQDQIRQAIQAATGKPYGEFNLDDEKKGMEAYINDQNSTNKKWNLYNMPSAYTILLTNKDSVPHVYYGD

SEQ ID NO: 3 - DNA, 674 bp, Lactobacillus sp.
gacaattcgaatccggtggtgcaagcggaacagttaaactggttatactacatgatgaatataggtagcattactgccaatgatcccaccgcaaactttgatggctatcgagtggacgctgtggacaatgtcgatgctgatttattaaatatagctgccgattatgccaaagatgcttataaaactaatcaaagtgatgctaatgccaacaaacatttatcaatattagaagattgggataataatgatccggcttatatcaaagcacatggaaataatcagttaactatggatttcccagcacatttagcaattaaatattcattaaatatgccagtaagtcaacgaagtgggctggaaccagagctcacaaccagtttagttaacagaactggtgatgattctactgaaaatgtcgcacagccaaactatacttttattagggctcacgatagtgaagtgcaaacaatcatcgcacaaattatcaaagataaaatcaaccctaactctgacggattaacagttactcccgatgaaataagtcaggcctttaaaatatataatgcagatgaattaaagactgataaacaatatactttttataacatgccctctgcctatactattttgctaaccaataaagatacagtaccccacctctattacggcgacat

SEQ ID NO: 4 - protein, 224 aa, Lactobacillus sp.
DNSNPVVQAEQLNWLYYMMNIGSITANDPTANFDGYRVDAVDNVDADLLNIAADYAKDAYKTNQSDANANKHLSILEDWDNNDPAYIKAHGNNQLTMDFPAHLAIKYSLNMPVSQRSGLEPELTTSLVNRTGDDSTENVAQPNYTFIRAHDSEVQTIIAQIIKDKINPNSDGLTVTPDEISQAFKIYNADELKTDKQYTFYNMPSAYTILLTNKDTVPHLYYGD

SEQ ID NO: 5 - DNA, 671 bp, Lactobacillus sp.
gacaacacgaacccggtggtgcaagctgagcagttaaattggttacactatttgatgaattttggtagcattacagcaaacgattctgctgctaattttgatgagatacgtgtcgatgctgtcgataatgttgacgctgatttactccagattgcagcagattatttcaaagctgcttatggtgttgataaaaatgacgcaacagcaaatcaacatctttcaattcttgaagattggagccataacgaccctgaatacgtgaaggattttggtaataatcaactcacaatggatgattacatgcatacccagttaatctggtcgttgactaaagatatgcgtatgcgtggtaccatgcaacgcttcatggactattacctcgtcaatcgcaatcacgatagtaccgaaaacactgccattccaaattacagctttgttcgcgcacacgatagtgaagtacaaacagtcattgctcaaattatttctgagttacatcccgacgtaaaaaatagtttggcaccaacagcagaccagctagccgaagcctttaaagtttataataacgatgaaaaacaggcggataagaaatatacacaatacaacatgcctagcgcctatgcgatgctgttaactaataaagatacagtaccccgcgtctactatggcgacat

SEQ ID NO: 6 - protein, 223 aa, Lactobacillus sp.
DNTNPVVQAEQLNWLHYLMNFGSITANDSAANFDEIRVDAVDNVDADLLQIAADYFKAAYGVDKNDATANQHLSILEDWSHNDPEYVKDFGNNQLTMDDYMHTQLIWSLTKDMRMRGTMQRFMDYYLVNRNHDSTENTAIPNYSFVRAHDSEVQTVIAQIISELHPDVKNSLAPTADQLAEAFKVYNNDEKQADKKYTQYNMPSAYAMLLTNKDTVPRVYYGD

SEQ ID NO: 7 - DNA, 746 bp, Lactobacillus sp.
gactcactatagggcgaattgggccctctagatgcatgctcgagcggccgccagtgtgatggatatctgcagaattcgcccttgacaattggaatccggtggtgcaagctgaacagttgaattggctacactatttgatgaatcttggaacgattacagctaatgatccagatgctaattttgacagcataagagttgacgctgtcgataatgtggatgcagatttgttagatattgcacgtgattactttaatgcagtatacaaggttaaccaaagtgatgttaatgctaataaacatatttctattttagaagattggagtggattagatcccaatgaggttgttaaaaatgggaatccacaattaacacttaacacaggggttcaaaattcattattaaatgctttgacaaaggggccaaataatcgttggggggatagactcattgattgataaatcaacaatgagatatccagataaggatggtaaaatccttattcctaattatagtttcgtacgtgcacacgatagtgaagttcaaggtattattggcaaatattaacagatcatacgtcagccgaatcaggtaataaattcacaaaggatccattaaaacaggcttggattattactatgctgaatcaagawtagactgttaaagaatattcgcactataatatggcgagtgcttatgcagcattgttaacaaattaagataccattcccaactctactacggcgact

SEQ ID NO: 8 - protein, 221 aa, Lactobacillus sp.
DNWNPVVQAEQLNWLHYLMNLGTITANDPDANFDSIRVDAVDNVDADLLDIARDYFNAVYKVNQSDVNANKHISILEDWSGLDPNEVVKNGNPQLTLNTGVQNSLLNALTKGPNNRWGIDSLIDKSTMRYPDKDGKILIPNYSFVRAHDSEVQGIIGKILTDHTSAESGNKFTKDQLKQALDYYYADQDKTVKEYSHYNMASAYAALLTNKNTIPNLYYGD

SEQ ID NO: 9 - DNA, 670 bp, Lactobacillus sp.
gataattcgaatccgatggtgcaagctgagcaattgaactggcttcactacatgatgaacattggtactatagctcagaacgacccaacagctaattttgacggttatcgtgttgatgcggttgataatgttgatgccgatctcttacaaattgctggtgattactttaaagctgcatacggtactggtaaaactgaggcaaacgcaaacaatcatatttcgatcttggaagattgggataataatgattctgcgtacattaaagcccacgggaacaaccaattgacaatggattttccagcacacttggctttgaaatacgccttgaacatgcctcttgccgcacaaagtggcctagaaccgctaattaatacaagtcttgttaagcgtgggaaagatgccacagaaaatgaagcacaaccaaactatgcctttatccgtgcccatgatagtgaagtgcagacagttattgcacaaattattaaggataaaattaacacaaaatcagacggcttaactgtaacaccagatgagattaagcaagctttcaatatttacaacgccgatgaattaaaagcagataaggaatatacagcatacaatattcctgcttcttacgctgtattgttgacaaacaaggatactgtgcccgcgtctactatggcgacct

SEQ ID NO: 10 - protein, 223 aa, Lactobacillus sp.
DNSNPMVQAEQLNWLHYMMNIGTIAQNDPTANFDGYRVDAVDNVDADLLQIAGDYFKAAYGTGKTEANANNHISILEDWDNNDSAYIKAHGNNQLTMDFPAHLALKYALNMPLAAQSGLEPLINTSLVKRGKDATENEAQPNYAFIRAHDSEVQTVIAQIIKDKINTKSDGLTVTPDEIKQAFNIYNADELKADKEYTAYNIPASYAVLLTNKDTVPASTMAT

SEQ ID NO: 11 - DNA, 6443 bp, Lactobacillus reuteri
  CDS (632)..(5962)
  modified_base (6311): a, c, g, t, unknown or other
gaattcgctaccgagatcttaaaggataaagacgttgaagtcgctcccgttgattcttcagcatatccagccaaagcttaccgtcctcgccattcagttatgagtttgaagaaggcagaagacactgggtttgagattatggattggcggactgcattgagtaagtttatagaggggattgaggagtaagatactggaaccggtttggattggatactgcttttttatgggcggcgcaataaagctagatctaactggaaaagactgcgaacaaaattgaaatttagtgtaagcagctaatatccttagtcaatgtagtataattgcaaattttttactaggtaagaaagtatattgtggaaatatttaagaatattgtcgttaccggtagagacaattttataagttctaactttgttcactatgttgttaacccttactaggaagttgaacatattacggttttagataagttaacttatactggcatttagtcaattctgatatctttgtttaaaattacaaatttgaactttgtttgaagaaaatgtgggaagaatttgaaaatttcctttaaaaaaattaaacatcatagtattataatatcgataattaaattgtttattctgacatgaaggagattaaaatggaaataaagaaacattttaagttgtataaaagtggtaaacaatgggtgacagcggcagttgctactgttgccgtttcaaccgcgcttctttacgggggagttgcgcatgctgatcaacaagttcagtcttccacaacccaagaacaaacttctactgtgaatgctgatactactaaaacagtaaatttagatactaatactgaccaaccagcccaaacaactgataaaaatcaagtagcaaatgacactactactaaccaaagtaaaactgatagtacatcaacaactgttaagaatcctacttttataccagtttctactttgtcttcatcagataatgaaaaacaaagtcaaaattataataagccggataatggaaactatggaaatgttgatgcagcttactttaataataatcaattgcatatttcaggatggcacgcaacaaatgcatctcaaggaacagatagtcgtcaggtgattgtacgtgatatcacaactaaaactgaattaggacgtactaatgtaacaaacaatgttttacgcccagatgttaaaaatgtccacaatgtttataacgctgataattctggattcgatgtcaacatcaacattgactttagtaagatgaaggactatcgtgattcaattgaaattgttagtcgatacagtggaaatggtaaatctgttgattggtggtctcaaccgattacctttgacaaaaataattacgcataccttgacacatttgaagttaaaaatggggaattgcatgcaacaggatggaatgctactaataaggcaattaactataaccaccattttgtaattttatttgatcgaacaaatggtaaagaagtgactcgtcaagaagttcgtgatggtcaatcgcgtccagatgttgctaaggtatatccacaagtagttggggcaaataactctggctttgacgtgacatttaatattggtgatctagattacactcatcaataccaaattcttagtcgttacagcaatgcagataatggcgaaggtgattatgttacttactggtttgctccacaatcaattgctcctgctaaccaaagtaatcagggttatttagattcatttgatattagtaaaaatggtgaagtgacagtaactggttggaatgctactgatctatctgaattacaaactaaccattatgtaattttatttgaccaaaccgctggtcaacaagttgcatctgcaaaagttgatctaatttcccgtccagatgttgcgaaagcttacccaacagtaaaaactgctgaaacttctggctttaaggtaacatttaaggttagtaatttacaaccaggtcatcaatatagtgtcgtaagccgtttttctgccgatgaaaacggtaatggtaatgataaacgtcataccgattactggtacagcccagtaaccttaaatcaaactgcttcaaatattgatactatcacaatgacatcgaatggattgcatattactggttggatggcaagtgataattcaattaatgaagcaactccatatgccattattcttaataatggtagagaggttactcgtcaaaaattaactttaattgcgcgtccagatgtagcagcagtatatccttcactctataacagtgctgttagtggatttgatactaccattaagttgactaatgctcaataccaggcgcttaatggtcaactacaagtattgttacgtttttctaaagctgttgatggtaatccaaacggcactaatactgtaacagatcaatttagtaagaattatgcaactactggtggaaactttgattatgtcaaagtaaacggcaatcaaattgaatttagtggctggcatgcaactaatcaatcaaatgataaaaattctcaatggattattgttttagttaatggtaaagaggtaaaacggcaattagttaatgatactaaggatggtgctgctgggttcaaccgtaatgatgtttacaaagtaaatccggctattgaaaatagtattatgtctgggttccaaggtattattactttacctgtaacagttaaggatgaaaatgttcagcttgttcatcgttttagtaatgatgcaaagactggtgaaggtaattatgttgatttctggtcagaagtaatgtctgttaaggacagcttccaaaagggtaatggtccgcttaatcaatttggtttacaaactattaacggccaacaatattatattgacccaacaactggccaacctcgtaagaatttcttattgcaaaatgggaacgattggatttactttgacaaagatactggtgctggaactaatgctcttaagttacaatttgataagggaacaatttctgctgatgagcaatatcgtcgaggaaatgaagcctatagttatgatgacaagagtattgaaaatgtaaatggttacttaacagctgatacttggtaccgaccaaaacaaatcttaaaggatggtactacttggactgactctaaagaaacagatatgcgcccaattttaatggtatggtggccaaatactgttacacaagcatattatcttaactacatgaagcaatatggtaatttattgccggctagtttaccaagcttcagtacagatgctgattctgctgaattaaatcattactccgagcttgttcaacaaaatatcgaaaagcggatcagtgagactggtagtactgattggttacgtacactaatgcatgagttcgttactaagaattctatgtggaataaggatagtgaaaatgtcgattacggtggtttgcaattacaaggtggattccttaagtatgtaaatagtgatcttactaaatatgcaaattcagattggcgtttaatgaaccgtacagctactaatattgatggtaagaactatggtggtgcggaattcttattagctaatgatattgataactcaaatccagttgttcaagctgaagaattaaactggctttactatttaatgaatttcggtacaattacaggaaataatcctgaagctaattttgatggtattcgagtggatgctgttgataatgtagatgttgacttattgagtattgcacgtgattactttaatgcagcatataacatggagcaaagtgatgccagtgctaataagcacattaatattttggaagattggggatgggatgatcctgcttatgtaaataagattggaaatcctcaattaacaatggatgatcgtttacgaaatgcaattatggatacattatcaggagcacctgataaaaaccaagcattgaataaattaattactcagtcattagtaaatcgtgctaatgataatactgaaaacgcggttattccaagctataattttgttcgagcacatgatagtaatgctcaagaccaaattcgtcaggctattcaagctgcaactggaaaaccatatggcgaatttaacttagatgatgaaaagaagggtatggaagcatatattaatgatcagaattctactaataagaagtggaatctttacaatatgccttctgcttatactattcttctaacaaataaagattcagttcctcgtgtttactatggagacctctaccaagatggtggtcaatatatggaacataaaacacgttactttgatactattactaacttattaaagacacgggttaaatatgttgccggtggacaaactatgagtgttgataagaatggtattcttacaaacgttcgttttgggaaaggcgccatgaatgctactgatactggtactgatgaaacaagaacagaaggtatcggtgttgtaattagtaacaatactaatttgaagcttaatgatggtgaatcagtagtgcttcatatgggagctgctcataagaatcaaaagtatcgtgctgtgatcttaacaactgaagatggtgttaagaattacactaatgatacagacgcaccagttgcatacactgatgctaatggtgaccttcactttactaatactaatttagatggtcaacaatatacagctgttcgtggatatgcaaatcctgatgtaacaggatatctagctgtttgggtaccagctggagcagcagatgatcaagatgcacgtactgcaccaagtgatgaggcccatactacaaagactgcttatcgctctaatgcagcccttgattctaacgttatttatgaaggattctctaacttcatttactggccaactactgaaagcgaacggactaatgtgagaattgcacaaaatgcggatctatttaagtcatggggaattactacctttgaattagctccacaatacaattcaagtaaagatggtacgttccttgattcaataattgataatggatatgcctttactgatcgttatgatttaggaatgagtactcctaacaagtatggatctgatgaagacttacgtaatgctttacaagccttacataaagctggtttacaagcaattgccgactgggttcctgatcaaatttataacttacctggtaaagaagctgtaacagtaacacgttcagatgatcacggtactacatgggaagtttcgccaataaagaatgttgtctatattacaaatacgattggtggaggtgaataccagaagaaatatggtggtgaattcttagacactcttcaaaaagaatatccacaattatttagtcaggtatatccagtaactcaaacgacaattgatcctagtgttaagattaaagagtggtctgctaaatactttaatggtactaatatccttcatcgaggtgctggatatgtattgcgctctaatgatggtaaatactataatcttggtacaagcactcaacaattcttaccgtctcaattatcagttcaagataatgaaggatatggatttgtaaaagaaggaaataattaccattactatgatgagaataaacagatggtaaaagatgcgtttattcaagatagtgttggtaattggtattacttcgataaaaatggtaatatggttgctaaccaaagtcctgttgaaattagtagtaatggagcttcaggaacttaccttttcttgaacaatgggacatcattccgttctggattggtgaaaactgatgcaggtacgtactattatgatggcgatggccgaatggttcgtaatcaaacggtaagtgatggtgcgatgacatatgttcttgatgaaaatggtaaacttgttagtgaatcatttgattcatctgctactgaagcacacccattaaaacctggtgatttaaacggccaaaaataattacaatatgaaaattggaacttgtattttaccttctttgaaataatatagttctaattaagcagctcgcaccaagacttggtatgagctgctttttttggctctacaatatctggtgttgatatagaaatatcactttctataccaatatcagatttttgtttttaaactaaaaaagaggctcgccctctgatacaatgaaatcgccaaatcacatagtaaagaaggtaacctccatggataatgatacaagaactcttctcaatttaacagaccctcatttaaattttcctcatcattggcttaaatataaaacaattaaaaaagttcgggtggcacaaatatnctgtaccctttcttatacaccacgggncttgtccaaattgggggagtcattaatcgnggtcaaatcttaaaatatgggcttttatcaagctaaacacaatatggacaatttaaaactcaaccattaatgntg

SEQ ID NO: 12 - protein, 1777 aa, Lactobacillus reuteri
MKEIKMEIKKHFKLYKSGKQWVTAAVATVAVSTALLYGGVAHADQQVQSSTTQEQTSTVNADTTKTVNLDTNTDQPAQTTDKNQVANDTTTNQSKTDSTSTTVKNPTFIPVSTLSSSDNEKQSQNYNKPDNGNYGNVDAAYFNNNQLHISGWHATNASQGTDSRQVIVRDITTKTELGRTNVTNNVLRPDVKNVHNVYNADNSGFDVNINIDFSKMKDYRDSIEIVSRYSGNGKSVDWWSQPITFDKNNYAYLDTFEVKNGELHATGWNATNKAINYNHHFVILFDRTNGKEVTRQEVRDGQSRPDVAKVYPQVVGANNSGFDVTFNIGDLDYTHQYQILSRYSNADNGEGDYVTYWFAPQSIAPANQSNQGYLDSFDISKNGEVTVTGWNATDLSELQTNHYVILFDQTAGQQVASAKVDLISRPDVAKAYPTVKTAETSGFKVTFKVSNLQPGHQYSVVSRFSADENGNGNDKRHTDYWYSPVTLNQTASNIDTITMTSNGLHITGWMASDNSINEATPYAIILNNGREVTRQKLTLIARPDVAAVYPSLYNSAVSGFDTTIKLTNAQYQALNGQLQVLLRFSKAVDGNPNGTNTVTDQFSKNYATTGGNFDYVKVNGNQIEFSGWHATNQSNDKNSQWIIVLVNGKEVKRQLVNDTKDGAAGFNRNDVYKVNPAIENSIMSGFQGIITLPVTVKDENVQLVHRFSNDAKTGEGNYVDFWSEVMSVKDSFQKGNGPLNQFGLQTINGQQYYIDPTTGQPRKNFLLQNGNDWIYFDKDTGAGTNALKLQFDKGTISADEQYRRGNEAYSYDDKSIENVNGYLTADTWYRPKQILKDGTTWTDSKETDMRPILMVWWPNTVTQAYYLNYMKQYGNLLPASLPSFSTDADSAELNHYSELVQQNIEKRISETGSTDWLRTLMHEFVTKNSMWNKDSENVDYGGLQLQGGFLKYVNSDLTKYANSDWRLMNRTATNIDGKNYGGAEFLLANDIDNSNPVVQAEELNWLYYLMNFGTITGNNPEANFDGIRVDAVDNVDVDLLSIARDYFNAAYNMEQSDASANKHINILEDWGWDDPAYVNKIGNPQLTMDDRLRNAIMDTLSGAPDKNQALNKLITQSLVNRANDNTENAVIPSYNFVRAHDSNAQDQIRQAIQAATGKPYGEFNLDDEKKGMEAYINDQNSTNKKWNLYNMPSAYTILLTNKDSVPRVYYGDLYQDGGQYMEHKTRYFDTITNLLKTRVKYVAGGQTMSVDKNGILTNVRFGKGAMNATDTGTDETRTEGIGVVISNNTNLKLNDGESVVLHMGAAHKNQKYRAVILTTEDGVKNYTNDTDAPVAYTDANGDLHFTNTNLDGQQYTAVRGYANPDVTGYLAVWVPAGAADDQDARTAPSDEAHTTKTAYRSNAALDSNVIYEGFSNFIYWPTTESERTNVRIAQNADLFKSWGITTFELAPQYNSSKDGTFLDSIIDNGYAFTDRYDLGMSTPNKYGSDEDLRNALQALHKAGLQAIADWVPDQIYNLPGKEAVTVTRSDDHGTTWEVSPIKNVVYITNTIGGGEYQKKYGGEFLDTLQKEYPQLFSQVYPVTQTTIDPSVKIKEWSAKYFNGTNILHRGAGYVLRSNDGKYYNLGTSTQQFLPSQLSVQDNEGYGFVKEGNNYHYYDENKQMVKDAFIQDSVGNWYYFDKNGNMVANQSPVEISSNGASGTYLFLNNGTSFRSGLVKTDAGTYYYDGDGRMVRNQTVSDGAMTYVLDENGKLVSESFDSSATEAHPLKPGDLNGQK

SEQ ID NO: 13 - DNA, 6065 bp, Lactobacillus reuteri
  CDS (43)..(5355)
atcgataatcaaattgtttattttgatataaaggagattaaaatggaaataaagaaacattttaagttgtataaaagtggtaaacaatgggtgacagcggctgttgctactgttgccgtttcaaccgcgcttctttacgggggagttgcacatgctgatcaacaagttcagtcttccacaactcaagaccaaacttctactgtaaatactaatactactaaaacaatagctgcagatactaatgctgatcagccagctcaaacagctgataaaaatcaagcagcatcaaatgacactactaaccaaagtaaaactgatagtacttcaacaactgttaagaatcttacttctacaccagtttctactttgccatcaactgataatgaaaaacaaaatcaaaattataataagcatgataatggaaactatgggaatattgatactgcttactttagcaataatcaattgcatgtttcaggatggaatgcaacgaatgcatctcaaggaacaaacagtcggcaaattattgtgcgtgatatcacaaccaataatgaattaggtcgtactgatgtaacaaacaatgttgcgcgcccagacgttaagaatgttcataatgtttataacgctgataattctggatttgatattaatgtcaatattgaatttagcaagatgaaagattatcgggattcaattgaaattgttagtcgatacagtggaaacggtaaatctattgactggtggtcccaaccgatcacttttgacaaaaacaattatgcttatcttgatacatttgaagtgaaaaatggcgaattacatgcaaccggatggaatgctactaatagtgcaattaactataatcaccattttgtaattttatttgatcaaacgaatggtaaggaagtagcacgacaagaagttcgtgaaggccaatcacgcccagatgttgctaaggtatatccacaagtagttggtgctgacaactccggctttgatgtgacatttaatatcggtaatttagattatactcaccagtaccaagttcttagtcgttacagcaattctgataatggcgaaggcgataatgttacctactggtttaatccacaatccattgctcctgctaatcaaagtaaccagggttatctagactcatttgatattagtaaaaatggtgaagtaacagtgaccggatggaatgctactgacttgtcagaattacaaaataaccattatgtaattctatttgatcagacagcaggcaaacaagtagcatctgccaaggctgatttaatttcacgtccagatgttgcaaaggcttatccaacagtaaaaactgctgcaaattctggctttaaggtaacatttaaggttaatgatttacaaccgggtcaccaatatagcgttgtaagtcgtttctctgccgatgaaaatggtaatggtaatgataagcgtcatacagattactggtttagtccagtaacattaaaccagaatgcttcaaacattgatactattacaatgacatctaatgggttacatattggcagttggatggcaagtgataactcaattaatgaaacaactccatatgctattattctcaataacggtaaagaagttactcgtcaaaagatgagtttaactgcccgtccagatgtagcagcagtatatccttcactttataatagtgctgttagtgggtttgatactactattaaattgactaatgatcagtatcaagcgcttaatggtcaattacaagtattgttacgtttttctaaagctgctgatggtaatccaagtggtgataatactgtaactgatcaatttagtaaaaattatgcaactactggtggaaactttgattatgtaaaagtaaatggtaatcaagttgaatttagtggttggcatgcaactaaccaatcaaatgataaagattcacaatggattattgttttagttaatggtaaagaagtaaagcgtcaattagttaatgatactaaagagggggctgctggcttcaaccgaaacgatgtctacaaagtaaatccagctattgaaaacagttctatgtctggattccaaggcattattactttaccagtaacagttaagaatgagaatgttcagattgtccatcgttttagtaatgatgcaaagacaggtgaaggtagccatgttgatttctggtcagaagtaatgccagttaaggatagtttccaaaagggtaatggtccgcttaagcaatttggcttacaaactattaatggtcatcaatattatattgacccaatgactggccaacctcgcaagaacttcctattacaaaatggtaatgactggctttattttgataatgaaactggtgagggaactaatgcgttaaagaggcaatttgacggaggaacgatttctgctgatagtcagtatagaaagggtaatgaagcttatggttatgacaataagagcattgaaaatgttgatggctttttaacagctgatacttggtaccgaccaaaacaaattttaaaatggaccacctggacagattctaaagaaacagatatgcgaccgctcttaatggtttggtggccaaatactgtaacccaagcatattaccttaactacatgaaacaacatggaaacttattaccagctaatcttccattctttaattctgatgcagatccattagaattaaattattatgcagaaattgttcagcaaaatattgaaaagaagattagtcaaactggtaatactgactggttgcgaactttgatgcacgaatttgtatctaataatacaatgtggaataagaatagtgaaaatgaagactttggtgggttgcaattacaaggtggttttctaaagtacgttaatagtgataagacacctaatgctaattctaattggcgtattatgggtaggcagccagctaatattgacggaaatgggccaattggatcagaattcttattagctaatgacgttgataattctaatccagttgttcaagctgaacagttaaattggctacattacttattgaattttggaactattactgcaaatgatcctgatgctaattttgatagcattcgtgttgatgctgttgacaatgtagatgccgatttattagatatagctggtgattactttaatgcagtatatcattctcaaagtaatgataaaattgctaatgctcatattaatattcttgaggattggggtggccaagatccgtattatacgcaaagcatcggaactcctcaattatcgatggattataatttctcaactataagaagtgtgttagcatctaacactgcatcaatgactgatattattaagaattcattggtaaatcggagcttagataatgctgaaaacgtatcaattcctaattactcatttatccgtgcacatgataatggttcacaagatgatattaagcgtgcaatttcagatgtaaataatttaccatatggttcgaagtttaactttgagcaagagcaaaaggggattgaagcatacattgcagatcaaagtaatgttaataagaagtggaataattataatattccatcttcatatgctattatgttgactaataaggataccgttcctcgtgtatattatggtgatttatttactgatggtggtcagtatatggcacaaacaacgcgttattatcctgcacttacaagtcttttaaaggcacgtattaagtatgtagctggtggacaaacaatgtctgtcgataagaataatattttgactagtgttcgctttggtaaaggtgcgatgaatcctactgatatgggtgatagtttaactagaacatctggtgttggggtagttataagtaataatgataaattattattaagctcaaatgataaagttgtattacacatgggtgctgcacataagaatcagaaatttaaagcagtcttactaactactaatgatggtattcagagttttaatgatgacaatgcgcctgttgcatatactgatgctaatggtgacttggtcctttctggtaaagatattacgactgatggtgtaattcaacataatactgctgttaagggctatgctaatgctgatgttaaaggttatcttgcagtatgggttccagtaggtgccagtgtacaacaggatattagaacagcaccatcaggggtacaaagtgatggaaagtctgtttatcattcaaatgcagctctggattcaaatattatttttgaaggattctctaactttgtatattggccgacaaataattctgagcgtgcaaatgtaaaaatcgctcagaatactgacttatttaaggagttgggtattacttcatttgaattagctccacagtataattcaagtaaggatggcacattccttgattctcagattgataatggatatgcatttactgatcgctatgatctaggtatgagcattccaaataagtatggtagcgatactgatctaaggaatgctattaaagccttacataaggccggaattcaagcaatggctgattgggttcctgatcaaatttataatttaccaggtaaagaagttgttactgctactcgtgttgacgaacgtggaaatgattggaatgtagctcagattaaggattcactttatgttgctaatacaattggtggtggaaagtatcaagagcaatatggtggagctttccttgatcaattacaaaagcaatatccacaaatctttgaacgtaaacaaccttcaactggtgtagcaattgacccaagtactaagattaaacagtggtctgctaaatactttaatgggacaaatattttacatcgtggtgcagggtatgtattaagagataacggtggtaactactttagccttggaaatagtaataataaacagttattaccaaatcaattatcaggtaaggctgaaaatggctttgttgatgttaatgggaatactaaatactttacatcaaccggaattcctgtcacggatgcatttgttcaagacagtgtaggtaactggtactatattgataaaaatggtaatatgcttaaaaataccggttttgtagatattacgcgaaatggtcagacaggtacgtatctattcttaaataacggtatctcattccgatcaggattagttaaaattggtaatgatacttattactttgacggtaatggaaaaatggttcgtggccaatctattagtgatggtacgatgaattatactcttgataaggatggtaaattagttggcttgtattatgatccaagtagtcagaatccacatccaattactcaacaggatttaagtggtactaataagtagtttattaaaaatcaccaatagaagttgtctctacatcaaatggtgttgatatgaaaatataatactttataccattaaattggtctagtaagaatcatcctcacggatggttctttttagtttcgccgtttgtaaaattaagttagaaaaaataaaaagccatttgtgatagacttttgagtatccctaatcaaaagaaaggcaatcacaaatgacctataaacatcttaccacacgcgaattaactctcatagctgatttttggtatcaaggcactaaagcttatcgggctgctaaattacttcaacgtagtcaagaaaccatctatcgtgtttatcgtttcctcaataacggtaaaaccatcgaccaatatcttcagacttatcagcgacataaacgtcgttgtggtcggaagcagacccaactgccaactatcgaggttaactatatccatgcgcaaatcaaggctggttggactcctgatactattattggtcgtgatgagcacccgattagctgcagatactaatgctgatcagccagctcaaacagctgataaaaatcaagcagcatcaaatgacactactaaccaaagtaaaactgatagtacttcaacaactggtaagaatcttacttctacaccagttttctactttggcatcaactgataatggaaaacaaaatcaaaattataataagcatgatat

SEQ ID NO: 14 - protein, 1771 aa, Lactobacillus reuteri
MEIKKHFKLYKSGKQWVTAAVATVAVSTALLYGGVAHADQQVQSSTTQDQTSTVNTNTTKTIAADTNADQPAQTADKNQAASNDTTNQSKTDSTSTTVKNLTSTPVSTLPSTDNEKQNQNYNKHDNGNYGNIDTAYFSNNQLHVSGWNATNASQGTNSRQIIVRDITTNNELGRTDVTNNVARPDVKNVHNVYNADNSGFDINVNIEFSKMKDYRDSIEIVSRYSGNGKSIDWWSQPITFDKNNYAYLDTFEVKNGELHATGWNATNSAINYNHHFVILFDQTNGKEVARQEVREGQSRPDVAKVYPQVVGADNSGFDVTFNIGNLDYTHQYQVLSRYSNSDNGEGDNVTYWFNPQSIAPANQSNQGYLDSFDISKNGEVTVTGWNATDLSELQNNHYVILFDQTAGKQVASAKADLISRPDVAKAYPTVKTAANSGFKVTFKVNDLQPGHQYSVVSRFSADENGNGNDKRHTDYWFSPVTLNQNASNIDTITMTSNGLHIGSWMASDNSINETTPYAIILNNGKEVTRQKMSLTARPDVAAVYPSLYNSAVSGFDTTIKLTNDQYQALNGQLQVLLRFSKAADGNPSGDNTVTDQFSKNYATTGGNFDYVKVNGNQVEFSGWHATNQSNDKDSQWIIVLVNGKEVKRQLVNDTKEGAAGFNRNDVYKVNPAIENSSMSGFQGIITLPVTVKNENVQIVHRFSNDAKTGEGSHVDFWSEVMPVKDSFQKGNGPLKQFGLQTINGHQYYIDPMTGQPRKNFLLQNGNDWLYFDNETGEGTNALKRQFDGGTISADSQYRKGNEAYGYDNKSIENVDGFLTADTWYRPKQILKWTTWTDSKETDMRPLLMVWWPNTVTQAYYLNYMKQHGNLLPANLPFFNSDADPLELNYYAEIVQQNIEKKISQTGNTDWLRTLMHEFVSNNTMWNKNSENEDFGGLQLQGGFLKYVNSDKTPNANSNWRIMGRQPANIDGNGPIGSEFLLANDVDNSNPVVQAEQLNWLHYLLNFGTITANDPDANFDSIRVDAVDNVDADLLDIAGDYFNAVYHSQSNDKIANAHINILEDWGGQDPYYTQSIGTPQLSMDYNFSTIRSVLASNTASMTDIIKNSLVNRSLDNAENVSIPNYSFIRAHDNGSQDDIKRAISDVNNLPYGSKFNFEQEQKGIEAYIADQSNVNKKWNNYNIPSSYAIMLTNKDTVPRVYYGDLFTDGGQYMAQTTRYYPALTSLLKARIKYVAGGQTMSVDKNNILTSVRFGKGAMNPTDMGDSLTRTSGVGVVISNNDKLLLSSNDKVVLHMGAAHKNQKFKAVLLTTNDGIQSFNDDNAPVAYTDANGDLVLSGKDITTDGVIQHNTAVKGYANADVKGYLAVWVPVGASVQQDIRTAPSGVQSDGKSVYHSNAALDSNIIFEGFSNFVYWPTNNSERANVKIAQNTDLFKELGITSFELAPQYNSSKDGTFLDSQIDNGYAFTDRYDLGMSIPNKYGSDTDLRNAIKALHKAGIQAMADWVPDQIYNLPGKEVVTATRVDERGNDWNVAQIKDSLYVANTIGGGKYQEQYGGAFLDQLQKQYPQIFERKQPSTGVAIDPSTKIKQWSAKYFNGTNILHRGAGYVLRDNGGNYFSLGNSNNKQLLPNQLSGKAENGFVDVNGNTKYFTSTGIPVTDAFVQDSVGNWYYIDKNGNMLKNTGFVDITRNGQTGTYLFLNNGISFRSGLVKIGNDTYYFDGNGKMVRGQSISDGTMNYTLDKDGKLVGLYYDPSSQNPHPITQQDLSGTNK

SEQ ID NO: 15 - DNA, 2361 bp, Lactobacillus reuteri
  CDS (1)..(2361)
aatattgatggttacttaagttatactggttggtatcgtccttatggaacgagtcaagatggtaaaacatggtacgaaacaactgcaatggattggcgtccattactgatgtatatttggccaagcaaagatgttcaagcacaatttattaagtattttgttaataatggttatgagaatgctaattatggacttacagagtcctctgttgcttcctttagcaaggatactaatgctaatctcctcgatgtaactgcacaaaatcttcgttatgtaattgagcaaagtattgcagccaataaagggacaagtaagttagcaaatgatattaatagttttgctgcaacggttcctgaattatctgcatcatctgaattatcattgcaaagcatgccaaactatcgaccagatgaaagtggaactgttgatagtgatcaagtcatttttgttaataataattcaaaggatccccgtaaagggaacactggttatgcggacagcaactatcgcttaatgaacaggacgattaataatcaggccggaaataataatagtgataacagtccagaactccttgttggtaatgatattgataattcaaacccagtagtacaagctgaaaatcttaattgggaatactttttactaaattatggtaagttaatggggtataatccagacggtaattttgatggcttccgagttgatgctgctgataatattgatgcagatgtcttagatcaaatgggtcaattaatgaacgacatgtatcatacaaagggaaatcctcaaaatgcaaatgatcatttgagttataatgaaggttatcattctggggctgcacaaatgctaaatgaaaagggtaatcctcaattgtacatggattcaggcgaattctatacccttgagaatgttctcggacgtgcaaataaccgtgatagtatcggtaatttaattactaatagtgttgttaatcggcaaaatgatacaacagagaatgaagctacgccaaactggtcatttgtaactaaccatgatcaacgaaagaatttgattaatagattaattattaagggtcatcctaacattccggatattatgggttcagcttacaaagctgaatatgcaaatcaagcatggcaagaattctacgctgatcagaaaaagactaataaacaatatgatcaatataatgttccggctcagtatgcaattcttttgagcaataaagatacggttccgcaggtttactatggtgacctttataatgaaactgctcaatacatgcaagagaagtcaatttactatgatacaatcacgactcttatgaaggcccgtaaacaatttgttagtggtggtcaaacgatgactaaacttaacaataatttattagctagtgttcgatatggtaagggtgttgctgattctaatagcaatggtaccgataagcttagccgaacaagtgggatagccgtcttagttggtaatgatagtaatatggctcaacaaactgttgctattaatatgggtcgcgctcatgctaaccaacaatatcgaaatttaattgatactaccgaaaatggcttgacatatgatggagaaaatagtgaaaatccagccattttgacaactgatagtaatggtatcttaaaagtaacagttaaaggatacagtaacccatacgtaagtggttatcttggtgtttgggttccagtaatttctggtgatcaagatgttactacaagtgcaagtgatgttgttgctgataaagaaaagacttttgaatctaatgctgctcttgattctcatatgatctatgaagatttcagcttgttccaaccagaaccaactaatgttgagaatcatgcttacaatgtgattgctaaaaatgctaatctctttaatgatttaggcattactgatttttggatggctcctgcttacactccatttggaatgagtcgttataatgaaggatactcaatgacggatcgttacaatttaggtacgacagctaatccaacaaagtatggtagtggagaagagcttgcaaatacaattgctgcattgcataaagtaggattaaaagttcaagaagatattgttatgaatcagatgattggtttctctggtcaagaagcagtaacggttactcgaacaaataatcgtggaatgcagattcatgtaaatggtcaaacatatgcaaatcaaatttactttgcatatacaactggtggcggaaatggtcaagaaacttatggtggtaaataccttgccgaattacaaaagaactatcctgacctatttacgaccaaggcaatttcgacagaagttgtacctgatccaaccgttcgtattaat

SEQ ID NO: 16 - protein, 787 aa, Lactobacillus reuteri
NIDGYLSYTGWYRPYGTSQDGKTWYETTAMDWRPLLMYIWPSKDVQAQFIKYFVNNGYENANYGLTESSVASFSKDTNANLLDVTAQNLRYVIEQSIAANKGTSKLANDINSFAATVPELSASSELSLQSMPNYRPDESGTVDSDQVIFVNNNSKDPRKGNTGYADSNYRLMNRTINNQAGNNNSDNSPELLVGNDIDNSNPVVQAENLNWEYFLLNYGKLMGYNPDGNFDGFRVDAADNIDADVLDQMGQLMNDMYHTKGNPQNANDHLSYNEGYHSGAAQMLNEKGNPQLYMDSGEFYTLENVLGRANNRDSIGNLITNSVVNRQNDTTENEATPNWSFVTNHDQRKNLINRLIIKGHPNIPDIMGSAYKAEYANQAWQEFYADQKKTNKQYDQYNVPAQYAILLSNKDTVPQVYYGDLYNETAQYMQEKSIYYDTITTLMKARKQFVSGGQTMTKLNNNLLASVRYGKGVADSNSNGTDKLSRTSGIAVLVGNDSNMAQQTVAINMGRAHANQQYRNLIDTTENGLTYDGENSENPAILTTDSNGILKVTVKGYSNPYVSGYLGVWVPVISGDQDVTTSASDVVADKEKTFESNAALDSHMIYEDFSLFQPEPTNVENHAYNVIAKNANLFNDLGITDFWMAPAYTPFGMSRYNEGYSMTDRYNLGTTANPTKYGSGEELANTIAALHKVGLKVQEDIVMNQMIGFSGQEAVTVTRTNNRGMQIHVNGQTYANQIYFAYTTGGGNGQETYGGKYLAELQKNYPDLFTTKAISTEVVPDPTVRIN

SEQ ID NO: 17 - DNA, 4686 bp, Lactobacillus sp.
atggaattaaaaaggcattacaagatgtacaaggctggtaaaaaatgggtttttgctgcaattgccacaatctctataattgcaggattaaatacagtggcagtgacaacctatgctgccggcaataatgatccgcagcagaccactactcaaaatgcacctaacaacagtaacgatccgcaatctactactacgcagaatactgccaacaacagtaacgatccgcaatctactactacgcagaatactgccaacaacagtaatggtccacaatctactactacgcagaatactgccaacaatagtaatggtccacaatctactactacgcagaatactgccaataacagtaacgatccacaatctactactacgcagaatactgccaacaacagtaacgatccgcaatctactactacgcagaatactgccaacaatagtaatggtccacaatctactactacgcagaatactgccaacaacagtaacgatccgcaatctactactacgcaaaacactgccaacaacggtaatgatccacaatctactactggaaaagatacagttagtattgcagatattcaagttaaccaacctgttaatcttttaggaaagcaatcaactgtatctagtactggttataatgactctcacataaaaaatgtcaatgggaaaatctattttgttggtgataatggtcaggtcaagaaaaactttacagccataatcaatggacaatcactatatttcaataaaacaactggagaattggcttctaatgatgttcaatatgaaaatgggttagtaaaaataaacgatgttcataacgccgcttactctattgatccaacgggattcactaatgttaacggatttttaactgctaatagttggtatagacccaaatatatttacaaagatgggcaaaaatgggtggaatcaacctctcaagatatgcgtccccttttaatgacatggtggccagataaaaatactcaagtagcttatttacaatatatgcagaaaatgggcattttacccgctgacgtcactatatcaagtcaaaccaatcaatcagttttaaccaaagaatcatttattactcaagctgaaattgaaaaacagattggagtaacaaatggaaacactgattggctaaagaaagatatctctgattttgtaaattctcaaccaaattggaatatagatagtgaagccaaaggcacagaccatttgcaggggggagcacttttatatgttaataataagttaactccatatgcgaattctgattaccgcttgcttaaccgaacacttactaatcaacaggggcaagtaaaagatacttctaaacaaggcggttatgaaatgttacttgccaacgatgtggataattccaatccagtagttcaagcggaacagttaaactggttatactacatgatgaatataggtagcattactgccaatgatcccaccgcaaactttgatggctatcgagtggacgctgtggacaatgtcgatgctgatttattaaatatagctgccgattatgccaaagatgcttataaaactaatcaaagtgatgctaatgccaacaaacatttatcaatattagaagattgggataataatgatccggcttatatcaaagcacatggaaataatcagttaactatggatttcccagcacatttagcaattaaatattcattaaatatgccagtaagtcaacgaagtgggctggaaccagagctcacaaccagtttagttaacagaactggtgatgattctactgaaaatgtcgcacagccaaactatacttttattagggctcacgatagtgaagtgcaaacaatcatcgcacaaattatcaaagataaaatcaaccctaactctgacggattaacagttactcccgatgaaataagtcaggcctttaaaatatataatgcagatgaattaaagactgataaacaatatactttttataacatgccctctgcctatactattttgctaaccaataaagatacagtacctcgagtttattatggggatctttatagtgataatggcaattatatgtcagcccattctccttactatgatgcaataactacgttattaaaaacacgaatgaaatacgtatctggtggtcaaaacatgcgtatgcaatatatgcagggtgatgatatgcctgctaatagctataagggcgttttaacttcagttagatatggtaagggtgaaatgacagccgatgagcaaggtaattcagaaactcgtactcaaggaattggggtcattataagcaataatcctaatttaaaattagacagtaatgaccaagtggtattaaatatgggggcggcacatgaaaatcaaacttatcgccctgtattactaacaactaaagatggattgaaaaactatgattccgatagttctgtacctcaaaatgcattagtttcaaccaacgataagggacaactcatatttaaagctagttctattcagggagtaagtaatccgcaggtatctggttatttgtccgtgtgggtcccagtgggggcaaaggataatcaagatgctcggactgcaagcagttctcagccatcaactgatgggaaaacatatcattccaatgctgctttagactctcaagttatttacgaaggattttctaattttcaatcgattcctacaaatacagaagatttcactaatgtaaaaattgctcaaaacgctaacctgtttaagagcttgggaataacaagttttgaattagcccctcaatatcgttccagtaatgataatagttttctggattcggttgttcaaaatggctacgcatttactgatcgttatgatattgggtataatactccgacaaaatatggaactgttactcaattgctggatgcattaagggctttacatgccaacggaattcaagcgatcgatgactgggttcctgaccaaatatacaatttacctggtgaggaaattgtcgcagctcaaagaactaatggatctgggacatatgatcaagattctgttattgatgatacattatatgattctcacactgttggtggtggcgaatatcaagctaaatttggtggagcttttctaaacaagttaaagcagttgtatcctgatttatttaaagttaaacaaatttctactggtcaacctatgaatcctaatgaaagaattaccgagtggtcagcaaagtactttaatggtacaaatattcaaggaagaggcgcttggtatgtattaaaagactggggtaccaatcagtactttaatgtaagtaataaccagtttgttcccaaacaattcctaggtacagatacttatacaggctttaatgttacaaatgagggaactcagttttattctacgagtgggtataaagcccagaatacctttattcaggacggagacaactggtattactttgacaataatggctatatggtaactggtttacagaatataaatgggaataattactatttcttgcccaatggcattgaactacaagactcttatttattgaatgatgataccggtaaagaatattattatgcaagtaatggtaagcaaatctcaaatcgttattatccagatgctaacggcaattggagatatttcttcaatgatggttcaatggcaagaaatggattaaccactattgaacaaccagatgggcaaaaagtgatccaatattttgattccgatggtattcaattaaagggaaatgccgcaaaagataataatggtaatttaagatattttgacggtaatacaggtgatatggtcattaattcatttggagaacttcctgatggctcttggttataccttaatgataaggggattgccgttactggtaaacaggaaatcaatggtcaaacctactactttgatgcggatggcaagcaagtgaagaatgattttagagagttgcctgatggttcatggctttatcttaatgacaaggggattgccgttactggtaaacaggaaatcaatggtcaaacctactactttgatgcggatggcaagcaagtgaagaatgattttagagagttgcctgatggttcatggctttatcttaatgacaaggggattgccgttactggtaaacagggaatcaatggtcaaacctatgcagaggctaaaatcacagctgccgaaaatgctcatcaagctgccacagacgctgtgaataaagcccaagctgctcaatcgcctaacactagttcctctagttctagcgttagccaagctactaaacatcaattggcagttaaaactgctaaagctcaacttgctaaaactaaggctcaaattgctaagtatcaaaaggctttgaaaaaagccaaaactacaaaggccaaggctcaagctcgtaaaagtttgaagaaggccgagactagtttcagcaaagctgaacttaatttggcattattaaataataaagccgtaaaagctgcacaaactaaggttaataaggctaaggctcaagtcactaaataccaaaaggctttgaagaaagctaagactacaaaggctaagactcaagctcgtaaaaatttgaagaaggccaactctagtctgacaaaagctcaaaaagcattaactaaagtaattaaaaccaatatcaagtaa

SEQ ID NO: 18 - protein, 1497 aa, Lactobacillus sp.
  MOD_RES (284): Variable amino acid
MELKRHYKMYKAGKKWVFAAIATISIIAGLNTVAVTTYAAGNNDPQQTTTQNAPNNSNDPQSTTTQNTANNSNDPQSTTTQNTANNSNGPQSTTTQNTANNSNGPQSTTTQNTANNSNDPQSTTTQNTANNSNDPQSTTTQNTANNSNGPQSTTTQNTANNSNDPQSTTTQNTANNGNDPQSTTGKDTVSIADIQVNQPVNLLGKQSTVSSTGYNDSHIKNVNGKIYFVGDNGQVKKNFTAIINGQSLYFNKTTGELASNDVQYENGLVKINDVHNAAYSIDPXGFTNVNGFLTANSWYRPKYIYKDGQKWVESTSQDMRPLLMTWWPDKNTQVAYLQYMQKMGILPADVTISSQTNQSVLTKESFITQAEIEKQIGVTNGNTDWLKKDISDFVNSQPNWNIDSEAKGTDHLQGGALLYVNNKLTPYANSDYRLLNRTLTNQQGQVKDTSKQGGYEMLLANDVDNSNPVVQAEQLNWLYYMMNIGSITANDPTANFDGYRVDAVDNVDADLLNIAADYAKDAYKTNQSDANANKHLSILEDWDNNDPAYIKAHGNNQLTMDFPAHLAIKYSLNMPVSQRSGLEPELTTSLVNRTGDDSTENVAQPNYTFIRAHDSEVQTIIAQIIKDKINPNSDGLTVTPDEISQAFKIYNADELKTDKQYTFYNMPSAYTILLTNKDTVPRVYYGDLYSDNGNYMSAHSPYYDAITTLLKTRMKYVSGGQNMRMQYMQGDDMPANSYKGVLTSVRYGKGEMTADEQGNSETRTQGIGVIISNNPNLKLDSNDQVVLNMGAAHENQTYRPVLLTTKDGLKNYDSDSSVPQNALVSTNDKGQLIFKASSIQGVSNPQVSGYLSVWVPVGAKDNQDARTASSSQPSTDGKTYHSNAALDSQVIYEGFSNFQSIPTNTEDFTNVKIAQNANLFKSLGITSFELAPQYRSSNDNSFLDSVVQNGYAFTDRYDIGYNTPTKYGTVTQLLDALRALHANGIQAIDDWVPDQIYNLPGEEIVAAQRTNGSGTYDQDSVIDDTLYDSHTVGGGEYQAKFGGAFLNKLKQLYPDLFKVKQISTGQPMNPNERITEWSAKYFNGTNIQGRGAWYVLKDWGTNQYFNVSNNQFVPKQFLGTDTYTGFNVTNEGTQFYSTSGYKAQNTFIQDGDNWYYFDNNGYMVTGLQNINGNNYYFLPNGIELQDSYLLNDDTGKEYYYASNGKQISNRYYPDANGNWRYFFNDGSMARNGLTTIEQPDGQKVIQYFDSDGIQLKGNAAKDNNGNLRYFDGNTGDMVINSFGELPDGSWLYLNDKGIAVTGKQEINGQTYYFDADGKQVKNDFRELPDGSWLYLNDKGIAVTGXQEINGQTYYFDADGKQVKNDFRELPDGSWLYLNDKGIAVTGKQGINGQTYAEAKITAAENAHQAATDAVNKAQAAQSPNTSSSSSSVSQATKHQLAVKTAKAQLAKTXAQIAKYQKALKKAKTTKAKAQARKSLKKAETSFSKAELNLALFK

SEQ ID NO: 19 - DNA, 6166 bp, Lactobacillus sake
  CDS (608)..(5392)
  modified_base (12): a, c, g, t, unknown or other
sasctgbcmstnacgtthrrcntagacgtthracgtactggttcacacaatggattcggcaaactatcaatgattgcgatctgtccaggttgggctgcttcacgcgtcaaaccagtacggatcgcattgaccacgggtaataattgtagtgcgcgacggttgaaccgtgaccgactaatggtgattttttgcggcataaaggcggtcatcaagcgccaaaaacggcgttgtgattgaataccaagcgttgtttgtaaacacagtagcgccaacaatcgacagtcatcgattttaacgtgcgccacattacgccgttgcgtcacacaacgtgggcaatagcgctggtaaagcgactggcacagctgataaaaataatgatagttaccttgtaattcgtgacgaatttgtttaaacttaggatggttcaacatcgttaggaccccttttaagtttagtcacttatgaatctaactgtgttggacttttttgttaatttttttgtattattacaaactagcaccacgcgtatgtgttttattaataccacttaattaataacggggctttagcatgatttcaaataaaatagtgtgaaaggtagttttttatgttaaggaataattattttggagagactaaaacgcattataaattatataaatgcggtaagaactgggctgtcatggggatttcattatttccgctgggattagggatgctagttaccagccagccagtgtcagctgatgtgacagccaccagcacctcaagcagtgcagtgaggaccgatgcaatcagtgcaagtagtagcagtgcagcaaaggctgaaacggctgcgatcactactgcaggtgttgcaaatgctgattcacaaacatcagcagaagtaaccgctgactctacttctaccagccaagtggtaactaataattccaataatcaaaataatacagcacagccagccggtcaagaagcagccccggtatcagaggacacatcatctgatgatagtgagagaactacaccaacagttgcaaataatgataagccagcaattgattcagttgacacttcacaacctgcaactgcagcgccaaaagcagacactgatgtatcaacgctacaagtagatgcaactacgaagaccgattcagacataaaagaggatacaccaacagataagacaaccgatacaaagactgtgcaattaaccactgttgaaggaacgtccaagcaagtggtaacgacgccgaaggaagagagctcaactgacaaatcttcgtctgtggtttctaaacaaacagacaaaacgtctttgccaaccgtagcaacagcaacagcgacgacagtgtctaagattccttcagtgacaggtgattaccagtttgacgaaaagacgaagacttatacgttcacaggtaaagatggtcatcccgtaactgggttggtttacgcgaataatatcctgcaatactttgatgaaacgggtcatcaagtaaaaggtcaatacgttacaattgcaggtcatgtatattatttcgacccagccagcggcgctgcacaaacaggtgttaatcaaatcgatggtaagatggttgggtttaaatctgatgggtcacaaattacgtcaggtttttctaatgataacgccggaaattcttactactttgatgagtctggaaccatggtgacagggtggcaaactattgctggtaagacgtattactttgacaaagacgggcatctccgtaaggggtattccactattattgataatcaattgtactatttcgatttgaaaacaggagagtctgtttcaacaacgacgtccaatttcaaatctggcttgacatcacaaacggatgacacaacaccacataatagtgcggttaatatgtctaaggatagttttaccaccgttgatggattcttgacagctgagtcatggtatgtacctaaagatattcaaacatctgcgacggactggcgtgcatcaacgcctgaagatttccgtccgatcatgatgacttggtggccaacgaagcaaattcaagcagcgtatttgaaccatatggtctccgaaggattgttgtcatcagataagaagttctccgcaacggatgatcaaacgttgttgaaccaagcagcacacgcggttcaattgcaaattgaattgaagattcaacagacaaagtctgttgaatggttgcgaacaacgatgcacaatttcattaagtcacaaccaggatacaatgttactagtgaaacgccaagtaacgaccaccttcaaggtggcgcattaagctacattaacagtgttttgacgcctgatgcgaactcaaatttccgtttgatgaaccgtaatccaacacaacaagatggtacgcgtcattacaacactgatacatctgagggtggatatgagttgctgttagccaatgacgtggataattctaacccagttgttcaagcagaacaattgaactggttgtacttcttgacgcatttcggtgaaattgttaagaacgatccgtcagctaactttgatagtgttagagtggatgcggtagacaacgtggatgccgacctgctaaacattacagccgcttattttagagatgtgtatggcgtcgataaaaacgatttgacagctaatcaacatttgtctattttggaagattggggccacaatgacccattatatgtcaaggaccacggtagtgatcagttgacgatggatgattacatgcatacccaattgatttggtcattaacaaaaaatccagataatcgtagtgcgatgcgacgatttatggagtattatttggtcgaccgtgctaaggacaatacgtctgatccagcaattcctaattacagctttgtccgtgcacacgatagtgaagttcaaacggttatcggtgatattgttgcgaagttgtatccggatgttaaaaattcattgccatctatggaacaattggcggcagcctttaaggtatacgatgcggatatgaattctgttaataagaagtatacgcaatacaacatgcccgcagcgtatgccatgttactaacgaataaagacacaattccacgtgtttactatggtgatatgtatacggatgatggtcaatatatggcaactaagtcaccatattacgatgccatctcagcgttgttgaaagcccgtattaagtatgtggctggtggacaaaccatggctgtagataaacacgatatcttaacatcagttcgctttggtgatgggatcatgaatgcatctgataagggtagcacgacggcccgtacccaaggaattggcgtgattgtcagcaataatgatgcgttagcgttgaagggagacactgtgacccttcatatgggtatcgctcacgccaaccaggcataccgtgctttgttgttaacgacgacagatggactaatgaaatacacgtccgataatggcgcgccaattcgctatacggatgcaaatggtgacttgattttcactagcgcagacattaagggataccaaaacgttgaggtatccggattcttgtcagtgtgggtaccagtcggtgcatccgacacacaggatgcgcgcgcaacagggtctagcgctgcaaacaaaactggtgacaccttacattcaaatgcagcattggactcaaatgtgatttatgaaggtttttctaatttccaagagatgccaacagcccacgatgagtttacaaacgtaaagatcgctcaaaatgctgatttgtttaagtcatggggtgtgacaagtttccaacttgcaccacaatatcgttcaagtgatgacacatcatttttggattctattattaagaatggatatgcgtttacagaccgctatgacttgggctttaatacgccaacaaagtacggagacgttgacgacctagcagatgcaattagagcaatgcacagtgttggtattcaggtcatggctgactttgtccctgaccaaatttataatttgccaggtcaagaagtagttgctgttaatcgtactaataactttggtacaccaaaccaggattcagatctacaaaaccagttgtatgttacaaattcaaagggtggcggtgaataccaagctaagtatggtggtgagttcttggatcttttgcgtctggaacaccctgatttgtttacaacaaatcagatttcgactggtgtaccaatcgatgggtccacgaagattaaagaatggtctgcaaagtacttcaatggttctgacatccaaggtaagggcgctgattacgtattgaaggatggtgcatctcaagaatatttcaagattacgtctaatgcgaacgatgagtccttcttgccaaaacaatttatgaatcaagatgccatgactgggttcaccacagatgaaaagggcacaacttattattcaacaagtggttaccaagctaaacagtcgtttatccaaggtgatgatggacaatattattactttgatgcagacggatacatggtgacgggctcacaaaccattaatggtaagcaatattacttcttgccaaatggcgttgagttaagagaagcatttttacaaaatgcatctggtaacacggtttattatggcaagactggctcagcagttaagtctaaatatgtagtcgatcaaagcggtgtggcttattactttgatgtaaacggtaatatggtcgcagatcgtatgatgattcttgatggacacacgcaatatttctttgcgggtggttcacaagctaaggaccaatttttgattgggtcagatggtaacttacgttactttgaccaaggtagtggtaatatggttacaaatcgttttgcagtaaaccgaaacggggattggttctacttcaatggcgatggtatcgcgttgaagggttggcaaacaattgctggtaagacttatttctttgatgctgatggacgtcaagtcaaggctgccgctgacaaggctgctgctgaacaagccgctgctgacaaggctgccgctgaacaagccgctgctgacaaggctgccgctaaggataagcaaactcaagctgtagcttacgctgctaccaaggctaagaacaatattgatcaagctactacagctgatggcatcaatgatgcccaagcaactggtatcactgatattgataaccagcatgttcctggtacttctgttgataataaaaagcaagctgagaaggtaactgaagatatcaagaatgatccagataataagactttgcctgaagctatcgaattaccaaatacgggcgttgataagacagaaagtattactattaccggtgtagttatgctaatcctcactactatttttggtctgttgtttacaagtaaaaagcataaaaaagattagtgtagatagctataccaaagggagttaacataacatcgattattcagatatgaacttatttagggactataatttacaaataacccctatgcaacgctattaaaacaacccccgttatctattggacaggtaataggggttgtttttatgtttttttatggcagattgcaagaaataacttgaacaaatttagtaacgcagattacgcaaaaagatctcaatcggtgttcgccaatttaaacatttgagtggtcgggaattcaaataccagttaatttgaatcaattcatcatcggtaatctcatcaatcggttgacctttgggaatgaagcggcgtagtactcggttgcgattttcattactgcctcgttcatgtggcgaataagcgtgcgcaaagtacagcggaacaccggcctgttcttcaatcaaattgtagttggcaaattctttcccatggtcaacagtaagggtcttgagattatctcctaactggttagccaattctaaaatagccttggtcattgaagtactatctcgtccattaagccgtttaacgatggtaagccgactcttacgctcgacaaacgtagctactgcttgacctttacgttttccagataaaactgtatcagcttcgaagtgacctgaagtattacgatcagaaatttcagctggacgatcttcgatggaacgtccatgactaaaactaccacgggtttctttagctcgtttgcgacgaataccatggtcaggtaaatcagtcacattaat

SEQ ID NO: 20 - protein, 1595 aa, Lactobacillus sake
MLRNNYFGETKTHYKLYKCGKNWAVMGISLFPLGLGMLVTSQPVSADVTATSTSSSAVRTDAISASSSSAAKAETAAITTAGVANADSQTSAEVTADSTSTSQVVTNNSNNQNNTAQPAGQEAAPVSEDTSSDDSERTTPTVANNDKPAIDSVDTSQPATAAPKADTDVSTLQVDATTKTDSDIKEDTPTDKTTDTKTVQLTTVEGTSKQVVTTPKEESSTDKSSSVVSKQTDKTSLPTVATATATTVSKIPSVTGDYQFDEKTKTYTFTGKDGHPVTGLVYANNILQYFDETGHQVKGQYVTIAGHVYYFDPASGAAQTGVNQIDGKMVGFKSDGSQITSGFSNDNAGNSYYFDESGTMVTGWQTIAGKTYYFDKDGHLRKGYSTIIDNQLYYFDLKTGESVSTTTSNFKSGLTSQTDDTTPHNSAVNMSKDSFTTVDGFLTAESWYVPKDIQTSATDWRASTPEDFRPIMMTWWPTKQIQAAYLNHMVSEGLLSSDKKFSATDDQTLLNQAAHAVQLQIELKIQQTKSVEWLRTTMHNFIKSQPGYNVTSETPSNDHLQGGALSYINSVLTPDANSNFRLMNRNPTQQDGTRHYNTDTSEGGYELLLANDVDNSNPVVQAEQLNWLYFLTHFGEIVKNDPSANFDSVRVDAVDNVDADLLNITAAYFRDVYGVDKNDLTANQHLSILEDWGHNDPLYVKDHGSDQLTMDDYMHTQLIWSLTKNPDNRSAMRRFMEYYLVDRAKDNTSDPAIPNYSFVRAHDSEVQTVIGDIVAKLYPDVKNSLPSMEQLAAAFKVYDADMNSVNKKYTQYNMPAAYAMLLTNKDTIPRVYYGDMYTDDGQYMATKSPYYDAISALLKARIKYVAGGQTMAVDKHDILTSVRFGDGIMNASDKGSTTARTQGIGVIVSNNDALALKGDTVTLHMGIAHANQAYRALLLTTTDGLMKYTSDNGAPIRYTDANGDLIFTSADIKGYQNVEVSGFLSVWVPVGASDTQDARATGSSAANKTGDTLHSNAALDSNVIYEGFSNFQEMPTAHDEFTNVKIAQNADLFKSWGVTSFQLAPQYRSSDDTSFLDSIIKNGYAFTDRYDLGFNTPTKYGDVDDLADAIRAMHSVGIQVMADFVPDQIYNLPGQEVVAVNRTNNFGTPNQDSDLQNQLYVTNSKGGGEYQAKYGGEFLDLLRLEHPDLFTTNQISTGVPIDGSTKIKEWSAKYFNGSDIQGKGADYVLKDGASQEYFKITSNANDESFLPKQFMNQDAMTGFTTDEKGTTYYSTSGYQAKQSFIQGDDGQYYYFDADGYMVTGSQTINGKQYYFLPNGVELREAFLQNASGNTVYYGKTGSAVKSKYVVDQSGVAYYFDVNGNMVADRMMILDGHTQYFFAGGSQAKDQFLIGSDGNLRYFDQGSGNMVTNRFAVNRNGDWFYFNGDGIALKGWQTIAGKTYFFDADGRQVKAAADKAAAEQAAADKAAAEQAAADKAAAKDKQTQAVAYAATKAKNNIDQATTADGINDAQATGITDIDNQHVPGTSVDNKKQAEKVTEDIKNDPDNKTLPEAIELPNTGVDKTESITITGVVMLILTTIFGLLFTSKKHKKD

SEQ ID NO: 21 - DNA, 3448 bp, Lactobacillus fermentum
  CDS (2)..(3019)
attaatggccgcatttgttgtgacacagccacagtggaataaaacaagtgaagatgtgaatgatgatcatttgcaaggtggggcattaacatttgaaaataacggcgacacagacgctaattcggattatcgcctcatgaaccgcacgccaacaaatcagactggcgaacgcttgtaccacattgatgactcacttggtggttacgaattattgctggcaaatgacgttgacaattcaaatccacaagttcaggcagaacaattgaattggttgtactacttaatgcattttggggatattacagctgatgatccggacgcaaattttgatgccatacggattgatgcggtcgataatgtcgatgctgatttacttcaactagcagcccagtatttccgggatgcctatggcatggctacaactgacgcaacatcaaataagcatctttcaattcttgaggattggagccataacgatccggcgtatatgcaagcacacggcaatgatcaattaacgatggatgattatatgcacacacagttgatttggtcattaaccaagcccgaggcacaacgcgggaccatggcacgctttatggacttctatctcaccaaccgtgctaatgatgatacagaaaacacggcgcaacctagttactcgtttgtgcgtgcccatgatagcgaagtacaaacagtcattgctgagatcgtgacgaagctgcatccagaagcaggaaatgggttaatgcctacggaagaacaaatggcagaagcgtttaagatttacaatgcggaccaaaagaaggccgttaagacttacacacattacaatatgccatctgcatacgccatgctgttaacgaacaaggatgttattccacgaatttactatggtgacttgtacactgatgatgggcaattcatggcgacaaaatcaccttattttgatgcgatttcgaccatgttacaagcacgcacgaagtatgtagctggtggacagacgatggcggttgaccagcacgacgtcttgactagcgttcggtttggtaagggggccatgacggccaatgatttaggggatgctgagacccggactgagggtgtgggattaattattagcaacaacccaaagttgcaattgggacaacaagacaacgtggtgttacacatgggacttgcgcacgcgaatcaggcattccgcgcagtcgtactaacgaccgcgaccggattaaccatttataatgacgatgatgctccgattcgttataccgataataagggtgatttaattttcactaaccatgacgtatatggcgtgttgaatccacaagtgtcaggcttcttggcaatgtgggtgccaactggtgcaccagcgaaccaggatgcgcgatctactgcgtcaaccaacatgtcaacggatggatctgcctaccattctaatgcggctttggatagtcaagtaatctttgaatcattttcgaatttccaggctatgccaacaagtcatgacacatacaccaacgttgtgttagccaatcatgctgaccagttgcacgattggggaataacttcggtacagttagcaccacaataccggtcttcaaccgacggtacctttttagacgcgattattcaaaatggctatgccttcactgaccgttatgatttagggtttggtacgccaactaaatacggggatgatacggatttgcggaacgtcatcaaagcattgcatgcaaatggcatgcaagtaatggctgattttgtgccggatcaattgtatacattaccaggtaaggaattggtacaagtcacccgaacaaacaatatgggtgagccagatacgcattctgacatccaacatattttatatgtgacgagcactcgtggtggtggtgactatcagaaacagtacggtggtgagttccttgcacgattgcgtgaacgatacccagatttatttacgacacgtcaaatttcgaccggacaaacaattgatgattcagtaaaaattaaagaatggtcagctaagtatttgaatggtaccgcaattcaaggacgtggagctggctatgtgctgcgtgataatggtacaaatgcttattacaaggtgacagcaaatgacggtaatgtgaacttaccaaagcaattactcggccaaccggtgatgaccggattctatcacgaggcagatggttatcattttgaaacattgagtggtacgtcggccaaagatgcctttattatgggcgacgatggggcactgtattattttgatgatcagggtgttatggtaacgggtaagcaacgtgtgcaccaagatcagtatttcttcctgccaaatggtattgctttgacagatgctttcgtacaaactgctgatggtcaacgtcagtactatgataaaacaggtcgtctggtcattaatcaatatgtgactgaccaccaagcgaatgcgttccgggttgatgcagacggtaacgttgtccgcaatcaagctttgactgttgacggccatgaacaatatttcggcacaaacggtgtccaagcgaaagcagtgctcattcgaactgacgataatcaggcgcgctactacgaagccaatagtggtaatctcgtgaagcaacagtttattcttgatacagatggacattggttgtacgcggatgctgcaggtgacttggcacgcggacaaattacaattggccaagacacgttgtattttgatgataataatcaccaggtaaaagatgatttcgtctatgatactaacggtgtgcattattttaatggcacaacaggcgctgaaatcaaacaagattacgcgtttcatgatggcaaatggtactattttgatgatttgggacgaatggtaaccggcttgcagcgtattaatggtgagtatcgctattttgatgctaatggtgtgcaactaaagggcggtaccgtgaccgatccactaacgcaccaaacgtacacttttgatgcgaaaactggtgctggtacgttggtgacgatttaactgaataatggactagaaaagacgatcttgtatcgtcttttttagtttcgataactaaataagtgctcatttttgcattaggactcagaattagcgggcgcgcaagcgtcttttcgtgttaaacttattagtaattaatattttgaggagtctgttatatggcaacaattttagttgtagatgatgaaccgtcattggtgacgctactgtcatacaacctgactaaatcaggcttcgaggtcgtgactgctacctccggtgacgaggcacgaaatcagctggcaaatcatcctattgatttgatgctgctaggtgtcatgttgcctggtaagagtggcgttgacttaacacgagaactacgaggcgaacagaatcgtattccaattattatgattaccgccttggatgacgaagttgacaagattt

SEQ ID NO: 22 - protein, 1006 aa, Lactobacillus fermentum
LMAAFVVTQPQWNKTSEDVNDDHLQGGALTFENNGDTDANSDYRLMNRTPTNQTGERLYHIDDSLGGYELLLANDVDNSNPQVQAEQLNWLYYLMHFGDITADDPDANFDAIRIDAVDNVDADLLQLAAQYFRDAYGMATTDATSNKHLSILEDWSHNDPAYMQAHGNDQLTMDDYMHTQLIWSLTKPEAQRGTMARFMDFYLTNRANDDTENTAQPSYSFVRAHDSEVQTVIAEIVTKLHPEAGNGLMPTEEQMAEAFKIYNADQKKAVKTYTHYNMPSAYAMLLTNKDVIPRIYYGDLYTDDGQFMATKSPYFDAISTMLQARTKYVAGGQTMAVDQHDVLTSVRFGKGAMTANDLGDAETRTEGVGLIISNNPKLQLGQQDNVVLHMGLAHANQAFRAVVLTTATGLTIYNDDDAPIRYTDNKGDLIFTNHDVYGVLNPQVSGFLAMWVPTGAPANQDARSTASTNMSTDGSAYHSNAALDSQVIFESFSNFQAMPTSHDTYTNVVLANHADQLHDWGITSVQLAPQYRSSTDGTFLDAIIQNGYAFTDRYDLGFGTPTKYGDDTDLRNVIKALHANGMQVMADFVPDQLYTLPGKELVQVTRTNNMGEPDTHSDIQHILYVTSTRGGGDYQKQYGGEFLARLRERYPDLFTTRQISTGQTIDDSVKIKEWSAKYLNGTAIQGRGAGYVLRDNGTNAYYKVTANDGNVNLPKQLLGQPVMTGFYHEADGYHFETLSGTSAKDAFIMGDDGALYYFDDQGVMVTGKQRVHQDQYFFLPNGIALTDAFVQTADGQRQYYDKTGRLVINQYVTDHQANAFRVDADGNVVRNQALTVDGHEQYFGTNGVQAKAVLIRTDDNQARYYEANSGNLVKQQFILDTDGHWLYADAAGDLARGQITIGQDTLYFDDNNHQVKDDFVYDTNGVHYFNGTTGAEIKQDYAFHDGKWYYFDDLGRMVTGLQRINGEYRYFDANGVQLKGGTVTDPLTHQTYTFDAKTGAGTLVTI

SEQ ID NO: 23 - protein, 525 aa, Lactobacillus reuteri
WPNTVTQAYYLNYMKQHGNLLPASLPFFNADADPAELNHYSEIVQQNIEKRISETGNTDWLRTLMHEFVTKNSMWNKDSENVDYGGLQLQGGFLKYVNSDLTKYANSDWRLMDRTATNIDGKNYGGAEFLLANDIDNSNPVVQAEELNWLYYLMNFGTITGNNPEANFDGIRVDAVDNVDVDLLSIARDYFNAAYNMEQSDANANKHINILEDWGWDDPAYVNKIGNPQLTMDDRLRNAIMDTLSGAPDKNQALNKLITQSLVNRANDNTENAVIPSYNFVRAHDSNAQDQIRQAIQAATGKPYGEFNLDDEKKGMEAYINDQNSTNKKWNLYNMPSAYTILLTNKDSVPRVYYGDLYQDGGQYMEHKTRYFDTITNLLKTRVKYVAGGQTMSVDKNGILTSVRFGKGAMNATDTGTDETRTEGIGVVISNNTNLKLNDGESVVLHMGAAHKNQKYRAVILTTEDGVKNYTNDTDAPVAYTDANGDLHFTNTNLDGQQYTAVRGYANPDVTGYLAVWVPAGAADD

SEQ ID NO: 24 - DNA, 22 bp, Artificial Sequence
  Description of Artificial Sequence Synthetic primer
adrtcnccrtartanavnykng

SEQ ID NO: 25 - DNA, 22 bp, Artificial Sequence
  Description of Artificial Sequence Synthetic primer
gayaaywsnaayccnryngtnc

SEQ ID NO: 26 - DNA, 51 bp, Artificial Sequence
  Description of Artificial Sequence Synthetic primer
gatgcatgagctcccatgggcattaacggccaacaatattattattgaccc

SEQ ID NO: 27 - DNA, 77 bp, Artificial Sequence
  Description of Artificial Sequence Synthetic primer
atatcgatgggccccggatcctattagtgatggtgatggtgatgtttttggccgtttaaatcaccaggttttaatgg

SEQ ID NO: 28 - protein, 6 aa, Artificial Sequence
  Description of Artificial Sequence Synthetic 6xHis tag
HHHHHH

SEQ ID NO: 29 - DNA, 12 bp, Artificial Sequence
  Description of Artificial Sequence Synthetic nucleotide repeat motif
aaaacaaccccc

SEQ ID NO: 30 - DNA, 12 bp, Artificial Sequence
  Description of Artificial Sequence Synthetic nucleotide repeat motif
ggggttgttttt